(19) United States
(12) Patent Application Publication
(10) Pub. No.: US 2015/0164922 A1
(43) Pub. Date: Jun. 18, 2015

(54) USE OF TETRACYCLINE COMPOSITIONS FOR WOUND TREATMENT AND SKIN RESTORATION

(71) Applicant: Foamix Pharmaceuticals Ltd., Rehovot (IL)

(72) Inventors: Dov Tamarkin, Macabim (IL); Elana Gazal, Rehovot (IL); Rita Keynan, Rehovot (IL); Meir Eini, Ness Ziona (IL); David Schuz, Gimzu (IL)

(21) Appl. No.: 14/384,978

(22) PCT Filed: Mar. 14, 2013
(86) PCT No.: PCT/IB2013/001170

§ 371 Date: Sep. 12, 2014

Related U.S. Application Data
(60) Provisional application No. 61/780,074, filed on Mar. 13, 2013.
(60) Provisional application No. 61/779,953, filed on Mar. 13, 2013.
(60) Provisional application No. 61/748,603, filed on Jan. 3, 2013.
(60) Provisional application No. 61/611,232, filed on Mar. 15, 2012.

(51) Int. Cl. A61K 31/65; A61K 47/06; A61K 47/44; A61K 9/00; A61K 47/12; A61K 47/46; A61K 47/02; A61K 47/24; A61K 47/10

(52) U.S. Cl. CPC A61K 31/65; A61K 47/24; A61K 47/06; A61K 47/44; A61K 47/10; A61K 47/12; A61K 47/46; A61K 47/02; A61K 9/0014

(57) ABSTRACT

Methods of treatment and dosage regimes using hydrophobic gel or foam compositions comprising a tetracycline antibiotic for accelerating the return of skin integrity and or in treating or alleviating a disorder including a wound, burn, impetigo, acne, rosacea, a skin disease caused by a bacteria or a tetracycline antibiotic responsive disease, wherein the foam composition or gel is administered topically to a target area on a subject having the disorder and wherein the target area comprises an area of skin, or mucosa or an eye.

### FIELD OF THE INVENTION

This invention relates generally to wounds and burns, compositions for the treatment of wounds and burns and restoration of skin integrity and compositions for use in the restoration of skin integrity or acceleration of the restoration of the integrity of an area of broken skin or mucosa by topical application.

### BACKGROUND OF THE INVENTION

Wound is an injury to the body (as from violence, accident, or surgery) that typically involves laceration or breaking of a membrane (as the skin) and usually damage to underlying tissues (Merriam Webster Dictionary). Burns are injuries to tissues caused by heat, friction, electricity, radiation, or chemicals. Wounds and burns are often colonized by microbiologic pathogens, including Gram-positive bacteria, such as Staphylococcus aureus and/or Streptococcus pyogenes; and Gram-negative bacteria, e.g., Pseudomonas aeruginosa.

In normal skin, the epidermis (outermost layer) and dermis (inner or deeper layer) exists in a steady-state equilibrium, forming a protective barrier against the external environment. Once the protective barrier is broken, the normal (physiologic) process of wound healing is immediately set in motion. The classic model of wound healing is divided into three or four sequential, yet overlapping, phases: (1) hemostasis (not considered a phase by some authors), (2) inflammatory, (3) proliferative and (4) remodeling (Guo S and DiPietro L A. Factors affecting wound healing. J Dent Res. 2010; 89: 219-229.)

Upon injury to the skin, a set of complex biochemical events takes place in a closely orchestrated cascade to repair the damage. FIG. 1 lays out the biological processes that occur in the course of the wound healing process and the respective biological factors that are involved, including cells (e.g., granulocytes, macrophages, fibroblasts) and cytokines (Ather S, D S Chan and K G Harding, The biology of wound healing. EJHP Practice 2007; 13: 53-55). As shown in FIG. 1, the cell proliferation phase reaches its peak about 10 days, followed by the remodeling phase, which may take up to 300 days.

FIG. 2 demonstrates photographically that the time scale of wound healing including the remodeling of the skin tissue structure can take a full month. In many cases, the healing of a wound is imperfect; resulting in the formation of scars; and attempts to accelerate the healing process may results in elevating the incidence of scar formation.

When the wound or burns are bacterially infected, the above process becomes more challenging and may take longer; and scars are more often caused following improper treatment.

Impetigo involves wounds with an infection of the superficial layers of the epidermis, caused by Gram-positive bacteria Staphylococcus aureus and/or Streptococcus pyogenes. A potentially more serious strain of the bacterium S. aureus has emerged in recent years that is resistant to certain antibiotics (Methicillin-resistant S. aureus, or MRSA).

Despite the very common occurrence of skin infections, only a limited number of topical antibiotics are approved for the treatment of wounds and particularly infected wounds. Mupirocin (Bactroban, GSK) is an antibiotic, developed by GSK. Emerging resistance to mupirocin is becoming a concern. In coagulase-negative staphylococci isolates, mupirocin resistance rates are higher, ranging from 12.7% in Europe to 38.8% in the United States. Retapamulin (Altabax, GSK) is another topical antibiotic used for wound treatment. Fucidin (LEO Pharma) is effective in primary and secondary skin infections caused by sensitive strains of S. aureus, Streptococcus species and C. minutissimum, but is virtually inactive against Gram-negative bacteria.

These three products require 6-10 days of treatment to attain clinical improvement. For example, Altabax attained 85.6% clinical success after 7 days, vs. 52.1% effect of the respective placebo.

Additionally, the above products are available as ointments, which when applied require rubbing onto the lesion, which is frequently an infected wound, leading to pain and transfer of infectious organisms to other sites. An additional drawback of Bactroban and Fucidin is that they require treatment three times daily, which imposes inconvenience to the caregivers of the impetigo patients, who are mostly infants and young children, so a product that requires less applications is advantageous and likely to improve compliance.

Acne, including acne vulgaris and acne-rosacea (also termed “rosacea”) are skin diseases which involve infected lesions, including non-inflammatory and inflammatory lesions. Non-inflammatory acne lesions include blackheads (open comedones) and whiteheads (closed comedones). Open and closed comedones along with papules and pustules are referred to as papulopustular acne, a form of inflammatory acne. The more severe the disease is, it involves more infected, inflammatory lesions. Nodular acne is the most severe form of inflammatory acne. If improperly treated, inflammatory acne lesions can produce deep scarring.

Oral antibiotics e.g. erythromycin, clindamycin and minocycline are prescribed for the treatment of infected wounds and burns, skin and soft tissue infections, impetigo, acne and rosacea; however, their use is generally associated with multiple, often systemic, side effects. Oral antibiotic can eradicate bacterial pathogens, but in general they are not known to accelerate in the remodeling of the skin tissue structure and the desirable skin healing

Minocycline, a semisynthetic derivative of tetracycline, is primarily used to treat acne and rosacea. Oral minocycline therapy is effective, but the clinical use is limited because of adverse effects such as upset stomach, diarrhea, dizziness, unsteadiness, drowsiness, mouth sores, headache and vomiting.

There is still a need to have a product for the treatment of skin lesions that involve disruption of the integrity or the structure of the skin, with or without a microbial involvement, which quickly

### SUMMARY OF THE INVENTION

The present invention relates to the discovery that a short course of treatment using topical minocycline is sufficient to achieve surprising clinical results in the treatment of infected wounds:

1. Quick onset of clinical effect: 80% of the patients improved after 3 days of treatment.
2. Clinical success is achieved in 100% of the patients.
3. All MRSA infections were cured following 7 days of treatment.
4. Skin healing/skin structure correction: In many of the patients the wounds disappeared and the skin structure returned to normal within 3-7 days
5. No scar formation was noted, despite the accelerated healing of the wounds.

It is further surprising that such results were not associated with any drug related side effects.

It has now surprisingly been found, that the topical administration of a gel or a foamable composition comprising a minocycline provided effective drug delivery to an infected lesion site, leading to rapid clinical improvement of impetigo within three days of treatment.

It has also surprisingly been found, that the topical administration of a gel or a foamable composition comprising a minocycline provided restoration of skin integrity and acceleration of restoration of skin integrity, leading to rapid clinical improvement within three days of treatment and return to skin integrity within seven days.

In one or more embodiments there is provided a hydrophobic gel or foam composition comprising a tetracycline antibiotic, for use in the restoration of skin integrity or acceleration of the restoration of the integrity of an area of broken skin or mucosa by topical application of the gel or foam composition to target area on a subject comprising an area of broken skin or mucosa or an area of skin containing a skin lesion.

In one or more embodiments the gel or foam composition consists of a carrier comprising about 60% to about 99% by weight of at least one hydrophobic oil.

In one or more embodiments there is provided a method of treating or alleviating a disorder selected from the group consisting of a wound, a burn, impetigo, acne, rosacea, and a skin disease caused by a bacteria, comprising administering topically at least once daily for at least three days to a target area on a subject having the disorder a hydrophobic gel or foam composition comprising a tetracycline antibiotic wherein the target area comprises an area of skin, mucosa, or eye.

In one or more embodiments there is provided a method of restoring or accelerating the restoration of the integrity of an area of broken skin or mucosa comprising administering topically at least once daily for at least three days to a target area on a subject comprising an area of broken skin or mucosa, a hydrophobic gel or foam composition comprising a tetracycline antibiotic.

In one or more embodiments there is provided a method of treating or alleviating a disorder comprising administering topically at least once daily for at least three days to a target area on a subject having the disorder a hydrophobic gel or foam composition comprising a tetracycline antibiotic wherein the target area comprises an area of skin, mucosa, or eye.

In one or more embodiments the disorder is selected from the group consisting of a wound, a chronic wound, a burn, impetigo, acne, rosacea, an inflammation, an ulcer, and a skin disease caused by a bacteria. In an embodiment the disorder is a wound. In an embodiment the disorder is a chronic wound. In an embodiment the disorder is a burn. In an embodiment the disorder is impetigo. In an embodiment the disorder is acne. In an embodiment the disorder is rosacea. In an embodiment the disorder is an inflamation. In an embodiment the disorder is an ulcer. In an embodiment the disorder is a skin disease caused by a bacteria. In an embodiment the disorder is a skin disease caused by a fungus. In an embodiment the disorder is a skin disease caused by a virus.

In one or more embodiments there is provided a method of treating or alleviating a disorder selected from the group consisting of impetigo, acne, rosacea, and a skin disease caused by a bacteria, comprising administering topically at least once daily for at least three days to a target area on a subject having the disorder a hydrophobic gel or foam composition comprising a tetracycline antibiotic wherein the target area comprises an area of skin, mucosa, or eye.

In one or more embodiments there is provided a method of restoring skin integrity or accelerating the restoration of the integrity of an area of broken skin or mucosa comprising administering topically a hydrophobic gel or foam composition comprising a tetracycline antibiotic at least once daily for at least three days to a target area on a subject comprising an area of broken skin or mucosa or an area of skin containing a skin lesion.

In one or more embodiments there is provided a hydrophobic gel or foam composition comprising a tetracycline antibiotic, for use in the restoration of skin integrity or acceleration of the restoration of the integrity of an area of a skin or mucosal lesion comprising a broken skin or a damaged mucosa, by topical application of the gel or foam composition to said skin or mucosal lesion,

wherein the gel or foam composition consists of a carrier comprising about 60% to about 99% by weight of at least one hydrophobic oil.

In one or more embodiments there is provided hydrophobic gel or foam composition comprising a tetracycline antibiotic, for use in the restoration of skin integrity or acceleration of the restoration of the integrity of an area of broken skin or mucosa by topical application of the gel or foam composition to a target area on a subject comprising an area of broken skin or mucosa or an area of skin containing a skin lesion,

wherein the gel or foam composition consists of a carrier comprising about 60% to about 99% by weight of at least one hydrophobic oil.

### BRIEF DESCRIPTION OF THE DRAWINGS

FIG. 1: the biological processes that occur in the course of the wound healing process and the respective biological factors that are involved, including cells (e.g., granulocytes, macrophages, fibroblasts) and cytokines (Ather S, D S Chan and K G Harding, The biology of wound healing. EJHP Practice 2007; 13: 53-55)

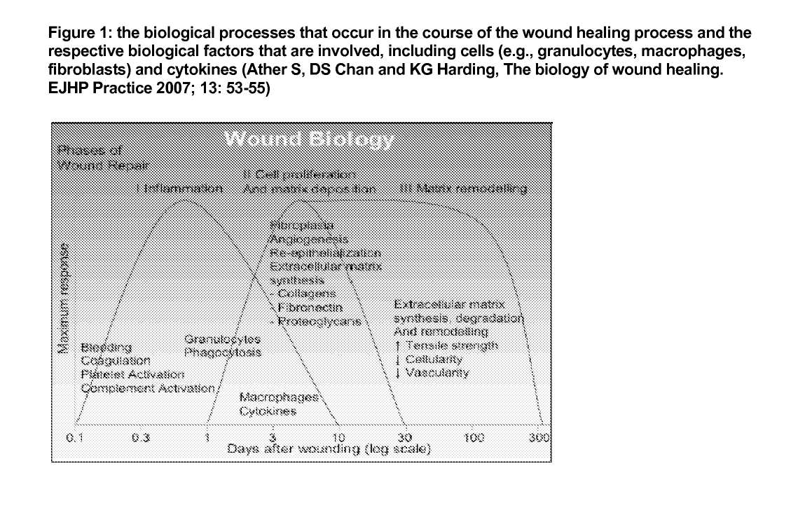

FIG. 2: Photographic demonstration of the time scale of wound healing including the remodeling of the skin tissue structure, which can take a full month.

FIG. 3: Pictorial examples of the baseline, Day 3 and EOT status of impetigo lesions following treatment with minocycline 1% and 4% topical foams. In these pictorial examples, the improvement is apparent as is also the restoration of visible, normal cutaneous topographic features, indicating the return of skin integrity.

FIG. 4: Improvement of a patient with acne within 6 weeks

### DETAILED DESCRIPTION OF THE INVENTION

The present invention relates to the discovery that a short course of treatment using topical tetracycline antibiotic is sufficient to achieve surprising clinical results in the treatment of infected wounds:

1. Quick onset of clinical effect: 80% of the patients improved after 3 days of treatment.
2. Clinical success is achieved in 100% of the patients.
3. All MRSA infections were cured following 7 days of treatment.
4. Skin healing/skin structure correction: In many of the patients the wounds disappeared and the skin structure returned to normal within 3-7 days
5. No scar formation was noted, despite the accelerated healing of the wounds.

It is further surprising that such results were not associated with any drug related side effects.

The topical minocycline composition used in the present invention comprises a lipophilic vehicle, in which the minocycline is suspended.

All % values are provided on a weight (w/w) basis.

By the term “about” herein it is meant that a figure or range of figures can vary plus or minus up to 10%. So in this embodiment if a figure of “about 1” is provided then the amount can be up to 1.1 or from 0.9. As will be appreciated by one of the art there is some reasonable flexibility in formulating compositions such that where one or more ingredients are varied successful formulations may still be made even if an amount falls slightly outside the range. Therefore, to allow for this possibility amounts are qualified by about. In one or more other embodiments the figures may be read without the prefix about.

It should be noted that the term “gel” means a jelly-like material that can have properties ranging from soft and fluid to hard and tough. Gels may be in liquid, semi-liquid, semi-solid or solid state. Solid gels are defined as a substantially diluted crosslinked system, which exhibits no flow when in the steady-state. By weight, gels are mostly liquid, yet they behave like semi-solids due to a three-dimensional crosslinked network of a solidifying, gelling or thickening agent within the liquid. It is the crosslinks within the fluid that give a gel its structure (hardness) and contribute to stickiness (tack). Depending on the amounts of gelling agent in a formulation the gel may be semi-solid with some limited flowability, such that when the semi-solid gel is placed in a tube and is inclined horizontally from a vertical position it will slowly flow from the vertical towards the horizontal or it may be a liquid gel where the amount of gelling agent or gelling effect is lower such that the gel structure or connections are weaker or loose so that when placed in a tube and tilted from a vertical position to the horizontal the gel readily flows and adapts to the horizontal position. The rheological properties of gels at different surface temperatures can influence the release and bioabsorption of drugs therefrom.

In one or more embodiments, the gel is stable and it retains its viscosity upon dispensing from a container, such as a tube, yet, it liquefies and spreads easily upon application of shear force, which can be mild, such as a simple rub. Further, while the gel is oily, it absorbs into the site of application, such as the skin or mucosa membrane, and after minutes the surface does not appear and or feel significantly oily or greasy.

The term “liquid gel” refers inter alia to the formulation after propellant is added (which prior to adding the propellant is a gel) or where the gel is loose or fluid or such that when subjected to gravity will pour or become liquid.

The term “waterless” or “water free” as used herein, means that the composition contains no, or essentially no, free or unassociated or absorbed water. Similarly, “substantially water free” or “substantially waterless” carriers contain at most incidental or trace amounts of water. In one or more embodiments, “substantially waterless” or substantially water free” means the composition contains about or less than 1%, about or less than 0.8%; about or less than 0.6%; about or less than 0.4%; about or less than 0.2%; about or less than 0.1%, about or less than 0.5%, about or less than 0.1%.

It should be noted that the term “surfactant” or “emulsifier” in the context herein refers to stand alone surfactants used to reduce surface tension between two substances or phases, which are also capable of stabilizing an emulsion of water and oil. Reduction of surface tension can be significant in foam technology in relation to the ability to create small stable bubbles. This is as opposed to the term surfactant which has often been loosely used in the art to include substances which do not function effectively as standalone surfactants to reduce surface tension between two substances or phases and which are also capable of stabilizing an emulsion of water and oil. For example, a surfactant as provided herein, does not include fatty acids, does not include fatty alcohols and does not include propoxylated lanolin oil derivatives. In the context of the present invention fatty acids and fatty alcohols are defined as foam adjuvants. Similarly, propoxylated lanolin oil derivatives in the context herein are defined as emollients.

“Standard surfactant” or “customary surfactant” or “stand alone surfactant” refers to customary non-ionic, anionic, cationic, zwitterionic, amphoteric and amphiphilic surfactants. Many standard surfactants are derivatives of fatty alcohols or fatty acids, such as ethers or esters formed from such fatty alcohols or fatty acids with hydrophilic moieties, such as polyethylene glycol (PEG). However, a native (non derivatized) fatty alcohol or fatty acid, as well as waxes are not regarded as a standard surfactant.

The term “co-surfactant” as used herein, means a molecule which on its own is not able to form and stabilize satisfactorily an oil in water emulsion but when used in combination with a surfactant the co-surfactant has properties which can allow it to help surfactants to create an emulsion and can boost the stabilizing power or effect of the surfactant. Examples include a fatty alcohol, such as cetyl alcohol or a fatty acid such as stearic acid. Cetyl alcohol is a waxy hydrophobic substance that can be emulsified with water using a surfactant. Some substances may have more than one function and for example, fatty alcohols can in some formulations act as a co-solvent. In certain circumstances, a co-surfactant can itself be converted into a surfactant or soap by, for example, adding a base, such as, triethanolamine to a fatty acid like stearic acid.

The term “viscosity modifying agent” in the context of the present invention is an agent which, when added to a hydrophobic oil, facilitates the creation of a hydrophobic breakable vehicle in the form of a breakable gel or breakable foam. In one or more embodiments the viscosity modifying agent is a “foamer complex” comprising a fatty alcohol, a fatty acid and/or a wax.

The term “breakable” refers to a unique property of the gel or the foam wherein the gel or foam is stable upon dispensing from a container, yet breaks and spreads easily upon application of shear or mechanical force, which can be mild such as a simple rub.

It should be noted that the term a “polyol”, as used herein, is an organic substance that contains at least two hydroxy groups in its molecular structure.

The identification of a “solvent,” as used herein, is not intended to characterize the solubilization capabilities of the solvent for any specific active agent or any other component of the foamable composition. Rather, such information is provided to aid in the identification of materials suitable for use as a part in the foamable composition described herein.

It should be noted that the term “a method of treating a disease or a disorder” as provided throughout the specification is interchangeable with the term “use of the composition as a medicament for treatment of a disease”. It should be noted the term a disease is used interchangeably with the term disorder.

It should be noted that the term “substantially free of” an ingredient as provided throughout the specification is intended to mean that the composition comprises less than about 0.5% by weight (e.g., less than about 0.2% by weight, less than about 0.1% by weight, less than about 0.05% by weight, less than about 0.01% by weight, less than about 0.001% by weight, or 0% by weight) of an ingredient.

The term “surfactant free” or emulsifier free” or “non-surfactant” composition means compositions which comprise no or negligible levels of surface active agents. Where a formulation includes insignificant or de minimis amounts of surface active agents it is considered to be essentially surfactant free.

The term “substantially surfactant-free” relates to a composition wherein the ratio between the viscosity-modifying agent and the surfactant is between 10:1 or 5:1; or between 20:1 and 10:1 or between 100:1 and 20:1. In additional embodiments, the term relates to a composition that contains a total of less than about 0.4% of a surfactant selected from the group consisting of customary non-ionic, anionic, cationic, zwitterionic, amphoteric and ampholytic surfactants.

Preferably, the composition comprises less than about 0.2% by weight of a standard surfactant or less than about 0.1%; or less than 0.05%.

By de minimis is meant so minor as to merit disregard.

The term “hydrophobic gel composition” or “hydrophobic foam composition” or “hydrophobic composition” is intended to mean that the composition has a low solubility in water. In an embodiment, 100 to 1000 parts of water are needed to dissolve or render miscible 1 part of composition. In an embodiment, 1000 to 10,000 parts of water are needed to dissolve or render miscible 1 part of composition. In an embodiment, more than 10,000 parts of water are needed to dissolve or render miscible 1 part of composition.

By “regular basis” is meant a repeated or repeatable interval of time which can be by way of illustration, a part of a day, daily, alternate daily, twice weekly, weekly, fortnightly, monthly or some other repeated or repeatable interval for an appropriate period of time wherein a dose is to be applied. In this connection the repeat applications will be according to the needs of the subject and the disease or disorder. In some circumstances as little as three repeat doses may be required in other cases, between 3 and 14, in other cases between 14 and 28, in other cases between 28 and 50, in other cases between 50 and 75, in other cases between 75 and 100 and in other cases such as where prolonged treatment or a long period of maintenance dosing is needed as many as one two or three hundred repeat doses may be needed.

The term safe in the context herein means having no or essentially no systemic or dermal adverse events.

The term tolerable or enhanced tolerability in the context herein means having no or essentially no skin irritation symptoms such as pigmentation, erythema, dryness, peeling and itching.

By “essentially no” in the context of tolerability includes insignificant or de minimis occurrences of skin irritation events manifested in symptoms such as pigmentation, erythema, dryness, peeling and itching or events not connected with the application of topical tetracyclines.

By “essentially no” in the context of safety includes insignificant or de minimis occurrences of systemic or dermal adverse events or events not connected with the application of topical tetracyclines.

In one or more embodiments, there is provided a method for eradicating MRSA thereby curing patients, and preventing the surrounding infants and children from contracting resistant bacterial infections by applying topically an effective amount of a tetracycline gel, liquid gel or foam to an infected area of a patient in need. In one or more embodiments, the method involves applying a gel, liquid, gel or foam formulation topically to a target surface in need of treatment and breaking the gel or foam over the target site. In one or more embodiments, the method uses a dosage regime of twice daily for three days followed by a daily maintenance dose for one, two, three or more weeks according to the condition and response of the patient. In one or more embodiments, the method uses a dosage regime of twice daily for four days followed by a daily maintenance dose for one, two, three or more weeks according to the condition and response of the patient. In one or more embodiments, the method uses a dosage regime of twice daily for one week followed by a daily maintenance dose for one, two, three or more weeks according to the condition and response of the patient. In one or more embodiments, the method uses a dosage regime of twice daily for two weeks followed by a daily maintenance dose for one, two, three or more weeks according to the condition and response of the patient.

It was suprising to find that in patients with impetigo wounds that substantially or deeply broke the integrity of the skin, the integrity of the skin was restored within 7 days, with an onset of healing within 3 days. By “restoration of the skin integrity”, it is intended that for a given lesion of the patient, the skin has healed until a point where it is without crusts and without erythema. By “onset of healing”, is intended a change for the better in cutaneous topographic features of the skin and or the beginning of closing of a breach in skin integrity. For example, when the skin lesions started to show an improvement of the erythema or dryness or exudation or peeling or a reduction of the area of the lesions or a reduction in the crust when compared to the baseline. As can be seen in the results described in the Examples section it is particularly surprising that even erythemas disappeared within 7 days of treatment in patients with impetigo wounds.

To the naked eye one of the markers of a wound is a breach in skin integrity. Returning of skin integrity occurs during the latter stages. It is visibly demonstrated by contraction of the wound. Contraction, is defined as the centripetal movement of wound edges that facilitates closure of a wound defect and results in a decrease in wound size. The rate of contraction depends on many factors including the position size and shape of the wound. Wound healing time means the amount of time it takes for the skin and underlying tissues to meet and fuse after a discontinuation of their surface by trauma. It can take weeks or months depending on the nature and extent of the trauma. For example a simple knife cut on the skin can take two to three weeks to heal if there are no complications. How close the edges of skin are is a relevant factor and the further apart they are the longer the process takes. The process will also take longer if the wound is or becomes infected.

Treating a breach in skin integrity attributable a disorder is not the same as treating the disorder or disease itself. Treating a cause of a disorder or disease may remove the cause but it will not be expected to remove the consequences. For example if the cause is a bacteria or fungi merely eliminating the bacteria will prevent the problem from becoming worse but it will be the bodies natural healing mechanisms, which can then act to restore a breach in skin integrity. Whilst skin integrity is breached there is a risk of further or secondary infections. So there is a need for a treatment that can accelerate the return of normal skin integrity. Accelerating wound healing can prevent or reduce scarring. To the extent an agent or formulation comprising the agent, which is effective in accelerating a return to normal skin integrity can also have a second activity for example, an anti-microbial, or an anti-bacterial or an anti-viral or an antifungal effect then the agent can act in a two or three fold way, namely accelerating the return of skin integrity, and or eliminating any microbes, and or preventing their return, it can be an advantage. However, the skin integrity repair agent can be used in compositions to restore integrity where its property e.g as an antibacterial is not significant as the cause of the breach is e.g. a fungal infection or is not due to a disease or disorder.

In an embodiment the breach in skin integrity is not caused by a disease or disorder but is due to an external physical cause, such a breach caused by an instrument or projection or a sharp object.

In one or more embodiments there is provided a method for treating a breach in skin integrity, including administering topically, to a surface having the breach in skin integrity, a composition comprising a tetracycline antibiotic.

In one or more embodiments there is provided a method for improving a breach in skin integrity, including administering topically, to a surface having the breach in skin integrity, a composition comprising a tetracycline antibiotic, wherein an improvement is considered as restoration of normal cutaneous topographic features and or closing of the breach indicating return of skin integrity.

In one or more embodiments the treatment effect or improvement is due to the presence of the tetracycline. In one or more other embodiments one or more formulation components also have a beneficial effect and add to the treatment effect or improvement. In one or more embodiments the treatment effect or improvement is due to the combination of the carrier composition and the tetracycline. In one or more embodiments the treatment effect or improvement due to the combination is synergistic.

In one or more embodiments the method involves applying a topical tetracycline composition to an area of skin having one or more breaches in skin integrity twice daily for seven days. In one or more other embodiments the application is once daily for seven days. In other embodiments the application is thrice daily for six days, or thrice daily for five days, or thrice daily for four days, or thrice daily for three days. In still other embodiments the application is twice daily for six days, or twice daily for five days, or twice daily for four days, or twice daily for three days.

I In one or more embodiments, the restoration of skin integrity is achieved within seven days. By within seven days includes the seventh day. In one or more embodiments, the restoration of skin integrity is achieved within seven days on at least about 25% of the lesions. In one or more embodiments, the restoration of skin integrity is achieved within seven days on at least about 50% of the lesions. In one or more embodiments, the restoration of skin integrity is achieved within seven days on at least about 75% of the lesions. In one or more embodiments, the restoration of the skin integrity is achieved within 7 days with onset of healing being within 3 days. In one or more embodiments, the integrity of the skin is fully restored within 7 days or less. In one or more embodiments, the restoration of the skin integrity is achieved within 3 days. In one or more embodiments, there is provided a restoration of the skin at a more rapid rate than would occur simply by removal of the cause of the lesion and then allowing the skin to heal.

I In one or more embodiments the onset of healing is observed within three days. By onset of healing is intended a change for the better in cutaneous topographic features of the skin and or the beginning of closing of a breach in skin integrity.

I In one or more embodiments, the restoration of the skin integrity concerns impetigo wounds. In one or more embodiments, the restoration of the skin integrity concerns acne wounds. In one or more embodiments, the restoration of the skin integrity concerns skin wounds or skin breaks.

In one or more embodiments, the treatment accelerates the restoration of skin integrity. By “acceleration” of the restoration of skin integrity it is intended that restoration of the skin is achieved at a more rapid rate than would occur by the removal of the cause of the lesion and allowing the skin to heal. By way of a non limiting example in the case of a skin breach which is caused by bacteria, it is intended that restoration of the skin is achieved at a more rapid rate than would occur by simply killing the bacteria and allowing the skin to heal. In one or more embodiments the acceleration is at least a 20% improvement in the healing time. In other embodiments it is at least a 30% improvement in the healing time. In further embodiments it is at least a 50% improvement in the healing time. In further embodiments it is at least a 60% improvement in the healing time. In further embodiments it is at least a 70% improvement in the healing time. In further embodiments it is at least a 80% improvement in the healing time. In further embodiments it is at least a 90% improvement in the healing time. In further embodiments it is at least a 100% improvement in the healing time.

In one or more embodiments there is provided a hydrophobic gel or foam composition comprising a tetracycline antibiotic, for use in the restoration of skin integrity by topical application of the foam composition to an area of skin containing a skin lesion.

In one or more embodiments there is provided a method of restoring the integrity of an area of skin containing a skin lesion, which method comprises topical application to said area of a hydrophobic gel or foam composition comprising a tetracycline antibiotic.

In one or more embodiments there is provided a hydrophobic gel or foam composition comprising a tetracycline antibiotic for use in treating a disorder selected from the group consisting of a wound, a burn, impetigo, acne, rosacea, and a skin disease cased by a bacteria, wherein the hydrophobic gel or foam composition is administered topically at least once daily for at least three days to the skin, mucosa, or eye. In further embodiments it is administered at least once daily for seven days. In other embodiments the hydrophobic gel or foam composition is administered topically at least twice daily for at least seven days to the skin, mucosa, or eye. In certain embodiments the hydrophobic gel or foam composition is waterless and does not comprise a silicone other than cyclomethicone. In other certain embodiments the hydrophobic gel or foam composition is waterless and does not comprise a polyethylene gelling agent or polyethylene homopolymer or polyethylene copolymer. In one or more embodiments a minocycline antibiotic is the sole active ingredient present in the composition.

In one or more embodiments there is provided a method of treating a wound or a burn, comprising the steps of:
(a) providing a therapeutically effective amount of a therapeutic hydrophobic breakable composition consisting of a carrier comprising about 60% to about 99% by weight of at least one hydrophobic oil; and a tetracycline antibiotic, suspended in the carrier; and
(b) applying the therapeutic substance at least once to outer surface of a wound or a burn;
   wherein the duration of treatment is such that an improvement of the wound or the burn is attained within 7 days of application.

In an embodiment the carrier comprises about 60% to about 99% by weight of at least one hydrophobic oil and a viscosity-modifying agents. In one or more embodiments the solvent is tested individually for compatibility with a tetracycline antibiotic and is only used if it passes a compatibility test. In one or more embodiments the viscosity-modifying agent is:
a. a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax; or
b. selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, pentatriacontanoic acid, a fatty acid, a hydroxy fatty acid, 12-hydroxy stearic acid, a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof

In one or more embodiments the therapeutic hydrophobic breakable composition is a gel. One or other embodiments the composition is packaged in an aerosol container to which is added a liquefied or compressed gas propellant the composition affords upon release from the container a breakable foam.

In one or more embodiments there is provided a hydrophobic gel or foam composition comprising a tetracycline antibiotic, for use in the restoration of skin integrity by topical application of the gel or foam composition to an area of skin containing a skin lesion, wherein the gel or foam composition consists of a carrier comprising about 60% to about 99% by weight of at least one hydrophobic oil; and wherein the a tetracycline antibiotic is suspended in the carrier. In one or more embodiments the gel or foam of claim 1, wherein the carrier comprises about 60% to about 99% by weight of at least one hydrophobic oil and a viscosity-modifying agents.

In one or more embodiments the hydrophobic oil is at a concentration of about 75% to about 90% by weight; or at least about 40% by weight; or at least about 50% by weight; or at least about 60% by weight; or at least about 70% by weight; or at least about 90% by weight.

In one or more embodiments the gel or foam further comprising at least one viscosity-modifying agent, selected from the group consisting of a fatty alcohol, a fatty acid and a wax.

In one or more embodiments the tetracycline antibiotic is:
a. a derivative of polycyclic naphthacene carboxamide; or
b. a compound selected from tetracycline, chlortetracycline, oxytetracycline, demeclocycline, doxycycline, lymecycline, meclocycline, methacycline, minocycline, rolitetracycline, chlorotetracycline and tigecycline;
wherein the tetracycline antibiotic a free base, or hydrate form, or a salt form or a complex form, or a derivative of said tetracycline antibiotic.

In one or more embodiments the tetracycline antibiotic is a tetracycline antibiotic having Log Kp equal to, or lower than about 0.2; or does not comprise any hydroxy group at Carbons 5, 6, and 7.

In one or more embodiments the tetracycline antibiotic is present in the composition in an amount ranging from about 0.001% to about 10%; or in an amount ranging from about 0.025% to about 6%; or in an amount ranging from about 0.5% to about 5% by weight of the carrier or in an amount ranging from about 0.1% to about 3%, by weight of the carrier composition. In an embodiment it is about 0.5%. In an embodiment it is about 1%. In an embodiment it is about 2%. In an embodiment it is about 3%. In an embodiment it is about 4%. In an embodiment it is about 5%. In an embodiment it is about 6%. In an embodiment it is about 7%. In an embodiment it is about 8%. In an embodiment it is about 9%. In an embodiment it is about 10%.

In one or more embodiments there is provided a method of restoring the integrity of an area of skin containing a skin lesion, which method comprises topical application to said area of a hydrophobic gel or foam composition comprising
a) about 48% to about 51% by weight of soybean oil;
b) about 23% to about 25% by weight of coconut oil;
c) about 4% to about 6% by weight of cyclomethicone;
d) about 0.5% to about 5% by weight of light mineral oil;
e) about 3% to about 4% by weight of cetostearyl alcohol;
f) about 2% to about 4% by weight of stearic acid;
g) about 2% to about 3% by weight of myristyl alcohol;
h) about 1% to about 3% by weight of hydrogenated castor oil;
i) about 1% to about 3% by weight of beeswax;
j) about 1% to about 2% by weight of stearyl alcohol;
k) about 0.5% to about 1.5% by weight of behenyl alcohol;
l) about 0.2% to about 0.5% by weight of modified (fumed) silica; and
m) about 1% to about 4% by weight of minocycline hydrochloride or doxycycline hyclate.

In an embodiment the tetracycline antibiotic is suspended in the composition.

In one or more embodiments there is provided a method of treating or alleviating a disorder selected from the group consisting of impetigo, acne, rosacea, and a skin disease caused by a bacteria, comprising administering topically at least once daily for at least three days to a target area on a subject having the disorder a hydrophobic gel or foam composition comprising a tetracycline antibiotic wherein the target area comprises an area of skin, mucosa, or eye.

In one or more embodiments the hydrophobic gel or foam composition comprises:
a) about 60% to about 99% by weight of at least one hydrophobic solvent;
b) at least one viscosity-modifying agent selected from the group consisting of a fatty alcohol, a fatty acid, and a wax; and
c) a therapeutically effective amount of a tetracycline antibiotic.

In one or more embodiments the hydrophobic foam is formed from the hydrophobic gel composition further comprising a propellant.

In one or more embodiments the disorder is impetigo.

In one or more embodiments the tetracycline antibiotic is selected from the group consisting of tetracycline, oxytetracycline, demeclocycline, doxycycline hyclate, lymecycline, meclocycline, methacycline, minocycline hydrochloride, rolitetracycline, chlorotetracycline, and tigecycline. In one or more embodiments the tetracycline antibiotic is minocycline hydrochloride. In one or more embodiments the minocycline hydrochloride is present in the composition at a concentration of about 1% by weight. In one or more embodiments the minocycline hydrochloride is present in the composition at a concentration of about 4% by weight.

In one or more embodiments the hydrophobic gel or foam composition is applied at a frequency selected from the group consisting of three times daily, twice daily, and once daily. In one or more embodiments the hydrophobic gel or foam composition is administered for a period selected from the group consisting of four days, five days, six days, seven days, eight days, nine days, ten days, eleven days, twelve days, thirteen days, and two weeks. In one or more embodiments a maintenance dose is applied thereafter at a frequency selected from the group consisting of every two days, three times a week, twice a week, and once a week. In one or more embodiments the maintenance dose is discontinued after a period selected from the group consisting of a week, two weeks, three weeks, four weeks, a month, two months, and three months.

In one or more embodiments the hydrophobic foam composition or gel is effective against methicillin-resistant S. aureus bacteria associated disorders.

In one or more embodiments at least about 40% of the impetigo lesions are cured after one week of treatment, wherein the hydrophobic foam composition or gel is administered twice daily. In one or more embodiments at least about 50% of the impetigo lesions are cured when observed one week after the end of the treatment. In one or more embodiments a decrease of at least about 50% in the total area of the impetigo lesions is obtained after one week of treatment, wherein the composition is administered twice daily. In one or more embodiments a decrease of at least 80% in the total area of the impetigo lesions is obtained when observed one week after the end of the treatment.

In one or more embodiments the hydrophobic gel or foam composition used in the method comprises:
a) about 48% to about 51% by weight of soybean oil;
b) about 23% to about 25% by weight of coconut oil;
c) about 4% to about 6% by weight of cyclomethicone;
d) about 0.5% to about 1.5% by weight of light mineral oil;
e) about 3% to about 4% by weight of cetostearyl alcohol;
f) about 2% to about 4% by weight of stearic acid;
g) about 2% to about 3% by weight of myristyl alcohol;
h) about 1% to about 3% by weight of hydrogenated castor oil;
i) about 1% to about 3% by weight of beeswax;
j) about 1% to about 2% by weight of stearyl alcohol;
k) about 0.5% to about 1.5% by weight of behenyl alcohol;
l) about 0.2% to about 0.5% by weight of modified (fumed) silica; and
m) about 1% by weight of minocycline hydrochloride or doxycycline hyclate.

In one or more embodiments the hydrophobic gel composition used in the method further comprises about 3% to about 25% by weight of propellant based on the total weight of the hydrophobic gel composition.

In one or more embodiments the hydrophobic gel or foam composition used in the method comprises:
a) about 48% to about 51% by weight of soybean oil;
b) about 23% to about 25% by weight of coconut oil;
c) about 4% to about 6% by weight of cyclomethicone;
d) about 0.5% to about 1.5% by weight of light mineral oil;
e) about 3% to about 4% by weight of cetostearyl alcohol;
f) about 2% to about 4% by weight of stearic acid;
g) about 2% to about 3% by weight of myristyl alcohol;
h) about 1% to about 3% by weight of hydrogenated castor oil;
i) about 1% to about 3% by weight of beeswax;
j) about 1% to about 2% by weight of stearyl alcohol;
k) about 0.5% to about 1.5% by weight of behenyl alcohol;
l) about 0.2% to about 0.5% by weight of modified (fumed) silica; and
m) about 4% by weight of minocycline hydrochloride or doxycycline hyclate.

In one or more embodiments the hydrophobic gel composition used in the method further comprises about 3% to about 25% by weight of propellant based on the total weight of the hydrophobic gel composition.

In one or more embodiments it is provided a method for retarding, arresting, or reversing the progression of a disorder in a mammalian subject in need thereof, the disorder selected from the group consisting of impetigo, acne, rosacea, and a skin disorder caused by a bacteria, the method comprising topically applying to the skin of the subject a hydrophobic foam composition or gel comprising a tetracycline antibiotic at least once a day for at least three days, thereby retarding, arresting, or reversing the progression of the disorder in the subject.

In one or more embodiments the hydrophobic gel or foam composition used in the method comprises:
d) Restart numbers about 60% to about 99% by weight of at least one hydrophobic solvent;
e) at least one viscosity-modifying agent selected from the group consisting of a fatty alcohol, a fatty acid, and a wax; and
f) a therapeutically effective amount of a tetracycline antibiotic.

In one or more embodiments the hydrophobic gel composition further comprises a propellant.

In one or more embodiments at least about 50% clinical success is observed after three days of treatment when the hydrophobic gel or foam composition is administered twice daily.

In one or more embodiments the hydrophobic gel or foam composition is safe and has high rates of clinical and microbiological responses when the hydrophobic gel or foam composition is administered twice daily.

In one or more embodiments the step of administering includes releasing the hydrophobic gel or foam composition and applying it onto the target area having the disorder, by collapsing and or spreading it on the target area using mild mechanical force thereby resulting in the hydrophobic gel or foam composition collapsing and being absorbed onto the a target area.

In one or more embodiments the hydrophobic gel or foam composition is absorbed within at least 120 seconds.

In one or more embodiments the method further comprises using a sterile applicator or prior to the steps of administering and/or collapsing and/or spreading, the hands of the person spreading are sterilized in order to avoid cross contamination.

In one or more embodiments a significant decrease in exudation score is obtained after three days of treatment, when the composition is administered twice daily.

In one or more embodiments a significant decrease in severity signs and symptoms is obtained after a week of treatment, when the composition is administered twice daily. In one or more embodiments the decrease is at least from severe to moderate or from moderate to mild or from mild to absent. In one or more embodiments the decrease is at least from severe to moderate or from moderate to mild or from mild to absent.

In one or more embodiments the composition has a shelf life of at least two years at ambient temperature.

In one or more embodiments the restoration of the skin integrity is achieved within seven days.

In one or more embodiments the onset of healing is achieved within three days.

### Therapeutic Hydrophobic Breakable Composition

In one or more embodiments there is provided topical therapeutic hydrophobic breakable composition consisting of a carrier comprising about 60% to about 99% by weight of at least one hydrophobic oil; and a tetracycline antibiotic, suspended in the carrier.

In one or more embodiments there is provided topical therapeutic hydrophobic breakable composition comprising:
a. a carrier comprising
   (i) about 60% to about 99% by weight of at least one hydrophobic oil
   (ii) a viscosity-modifying agents
b. a tetracycline antibiotic, suspended in the carrier

In an embodiment of the present invention the therapeutic hydrophobic breakable composition is a gel.

In an embodiment of the present invention, when the therapeutic hydrophobic breakable composition is packaged in an aerosol container to which is added a liquefied or compressed gas propellant the composition affords upon release from the container a breakable foam of at least good quality that breaks easily upon application of shear force. In one or more embodiments the propellant is about 3% to about 25% by weight of the composition. In one or more embodiments the propellant is a liquefied hydrocarbon gas propellant. In one or more embodiments it is hydrofluorcarbon propellant. In one or more embodiments the propellant is selected from the group consisting of propane, butane, iso butane and mixtures of any two or more thereof. In one or more embodiments the gel is contained in a canister to which is added a propellant and the foam is formed when the composition is released from the cannister.

### Hydrophobic Oil

In one or more embodiments, the at least one hydrophobic oil comprises or is selected from the group consisting of a mineral oil, a hydrocarbon oil, an ester oil, an ester of a dicarboxylic acid, a triglyceride oil, an oil of plant origin, an oil from animal origin, an unsaturated or polyunsaturated oil, a diglyceride, a PPG alkyl ether, an essential oil, a silicone oil, liquid paraffin, an isoparaffin, a polyalphaolefin, a polyolefin, polyisobutylene, a synthetic isoalkane, isohexadecane, isododecane, alkyl benzoate, alkyl octanoate, C12-C15 alkyl benzoate, C12-C15 alkyl octanoate, arachidyl behenate, arachidyl propionate, benzyl laurate, benzyl myristate, benzyl palmitate, bis(octyldodecyl stearoyl) dimer dilinoleate, butyl myristate, butyl stearate, cetearyl ethylhexanoate, cetearyl isononanoate, cetyl acetate, cetyl ethylhexanoate, cetyl lactate, cetyl myristate, cetyl octanoate, cetyl palmitate, cetyl ricinoleate, decyl oleate, diethyleneglycol diethylhexanoate, diethyleneglycol dioctanoate, diethyleneglycol diisononanoate, diethyleneglycol diisononanoate, diethylhexanoate, diethylhexyl adipate, diethylhexyl malate, diethylhexyl succinate, diisopropyl adipate, diisopropyl dimerate, diisopropyl sebacate, diisosteary dimer dilinoleate, diisostearyl fumerate, dioctyl malate, dioctyl sebacate, dodecyl oleate, ethylhexyl palmitate, ester derivatives of lanolic acid, ethylhexyl cocoate, ethylhexyl ethylhexanoate, ethylhexyl hydroxystarate, ethylhexyl isononanoate, ethylhexyl palmytate, ethylhexyl pelargonate, ethylhexyl stearate, hexadecyl stearate, hexyl laurate, isoamyl laurate, isocetyl behenate, isocetyl lanolate, isocetyl palmitate, isocetyl stearate, isocetyl salicylate, isocetyl stearate, isocetyl stearoyl stearate, isocetearyl octanoate, isodecyl ethylhexanoate, isodecyl isononanoate, isodecyl oleate, isononyl isononanoate, isodecyl oleate, isohexyl decanoate, isononyl octanoate, isopropyl isostearate, isopropyl lanolate, isopropyl laurate, isopropyl myristate, isopropyl palmitate, isopropyl stearate, isostearyl behenate, isosteary citrate, isostearyl erucate, isostearyl glycolate, isostearyl isononanoate, isostearyl isostearate, isostearyl lactate, isostearyl linoleate, isostearyl linolenate, isostearyl malate, isostearyl neopentanoate, isostearyl palmitate, isosteary salicylate, isosteary tartarate, isotridecyl isononanoate, isotridecyl isononanoate, lauryl lactate, myristyl lactate, myristyl myristate, myristyl neopentanoate, myristyl propionate, octyldodecyl myristate, neopentylglycol dicaprate, octyl dodecanol, octyl stearate, octyl palmitate, octyldodecyl behenate, octyldodecyl hydroxystearate, octyldodecyl myristate, octyldodecyl stearoyl stearate, oleyl erucate, oleyl lactate, oleyl oleate, propyl myristate, propylene glycol myristyl ether acetate, propylene glycol dicaprate, propylene glycol dicaprylate, propylene glycol dicaprylate, maleated soybean oil, stearyl caprate, stearyl heptanoate, stearyl propionate, tocopheryl acetate, tocopheryl linoleate, glyceryl oleate, tridecyl ethylhexanoate, tridecyl isononanoate, triisocetyl citrate, alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides, wheat germ oil, PPG-2 butyl ether, PPG-4 butyl ether, PPG-5 butyl ether, PPG-9 butyl ether, PPG-12 butyl ether, PPG-14 butyl ether, PPG-15 butyl ether, PPG-15 stearyl ether, PPG-16 butyl ether, PPG-17 butyl ether, PPG-18 butyl ether, PPG-20 butyl ether, PPG-22 butyl ether, PPG-24 butyl ether, PPG-26 butyl ether, PPG-30 butyl ether, PPG-33 butyl ether, PPG-40 butyl ether, PPG-52 butyl ether, PPG-53 butyl ether, PPG-10 cetyl ether, PPG-28 cetyl ether, PPG-30 cetyl ether, PPG-50 cetyl ether, PPG-30 isocetyl ether, PPG-4 lauryl ether, PPG-7 lauryl ether, PPG-2 methyl ether, PPG-3 methyl ether, PPG-3 myristyl ether, PPG-4 myristyl ether, PPG-10 oleyl ether, PPG-20 oleyl ether, PPG-23 oleyl ether, PPG-30 oleyl ether, PPG-37 oleyl ether, PPG-40 butyl ether, PPG-50 oleyl ether, PPG-11 stearyl ether, herring oil, cod-liver oil, salmon oil, cyclomethicone, a dimethyl polysiloxane, dimethicone, an epoxy-modified silicone oil, a fatty acid-modified silicone oil, a fluoro group-modified silicone oil, a methylphenylpolysiloxane, phenyl trimethicone and a polyether group-modified silicone oil. In some embodiments, the hydrophobic oil comprises or is selected from the group consisting of soybean oil, a coconut oil, a cyclomethicone, a light mineral oil, and mixtures thereof.

In one or more embodiments the solvent is tested individually for compatibility with a tetracycline antibiotic and is only used if it passes a compatibility test.

In one or more embodiments, the hydrophobic oil is at a concentration of about 75% to about 90% by weight. In one or more embodiments, the hydrophobic oil is at a concentration of at least about 40% by weight, at least about 50% by weight, at least about 60% by weight, at least about 70% by weight, at least about 90% by weight. In some embodiments, the hydrophobic oil is at a concentration of less than about 90% by weight, less than about 80% by weight, less than about 70% by weight, less than about 60% by weight, less than about 50% by weight.

### Viscosity Modifying Agent

The viscosity-modifying agent is selected from the group consisting of a fatty alcohol, a fatty acid and a wax.

In one or more embodiments, the viscosity-modifying agent is at a concentration of about 0.1% to about 22%, about 0.4 to about 18%, about 0.5% to 16%, about 0.6% to 14%, about 0.7% to 13%, about 0.8 to about 12%, about 0,9% to about 11%, about 1% to about 10%, about 10% to about 22% by weight. In one or more embodiments, the viscosity-modifying agent is a fatty alcohol having at least 12 carbon atoms in its carbon backbone. In one or more embodiments, the viscosity-modifying agent is a fatty acid having at least 12 carbon atoms in its carbon backbone.

In one or more embodiments, the viscosity-modifying agent is at a concentration of about 9.5% or about 8.5% or about 7.5% or about 6.5% or about 5.5% or about 4.5% or about 3.5% or about 2.5% or about 1.5%, about 7% or about 6% or about 5% or about 4% or about 3% or about 2% or about 1% or about 0.5%, or about 1.9%, or about 1.8%, or about 1.7%, or about 1.6%, or about 1.55 or about 1.4% or about 1.3% or about 1.2% or about 1.1%, or about 0.9% or about 0.8%, or about 0.7%, or about 0.6% or about 0.5% by weight of the composition or less than any of the aforesaid amounts.

In one or more embodiments, the fatty alcohol and/or fatty acid have a melting point of at least about 40° C.

NumberIn one or more embodiments, the fatty alcohol comprises or is selected from the group consisting of lauryl alcohol, myristyl alcohol, cetyl alcohol, stearyl alcohol, arachidyl alcohol, behenyl alcohol, tetracosanol, hexacosanol, octacosanol, triacontanol, and tetratriacontanol. In one or more embodiments, the fatty acid comprises or is selected from the group consisting of dodecanoic acid, tetradecanoic acid, hexadecanoic acid, heptadecanoic acid, octadecanoic acid, eicosanoic acid, docosanoic acid, tetracosanoic acid, hexacosanoic acid, heptacosanoic acid, octacosanoic acid, triacontanoic acid, dotriacontanoic acid, tritriacontanoic acid, tetratriacontanoic acid, and pentatriacontanoic acid.

In one or more embodiments, the carbon chain of the fatty alcohol or the fatty acid is substituted with a hydroxyl group.

In one or more embodiments, the fatty acid is 12-hydroxy stearic acid.

In one or more embodiments, the viscosity-modifying agent is a wax comprising or selected from the group consisting of a plant wax, carnauba wax, candelilla wax, ouricury wax, sugarcane wax, retamo wax, jojoba oil, an animal waxes, beeswax, a petroleum derived wax, a paraffin wax, polyethylene, and derivatives thereof.

n one or more embodiments, the viscosity-modifying agent is a combination comprising (i) at least one fatty alcohol and at least one fatty acid; or (ii) at least one fatty alcohol and at least one wax; or (iii) at least one fatty acid and at least one wax; or (iv) at least one fatty alcohol, at least one fatty acid, and at least one wax.

### Surface Active Agents

For clarification, in the context herein whilst the term “standard surfactant” or “customary surfactant” refers herein to customary non-ionic, anionic, cationic, zwitterionic, amphoteric and amphiphilic surfactants A fatty alcohol or a fatty acid and certain waxes are not regarded as a standard surfactant. However, in contrast, ethers or esters formed from such fatty alcohols or fatty acids can be regarded as a customary surfactant.

Surfactants of all kinds are undesirable in accordance with the present invention, as (i) they were found to cause degradation of the tetracycline antibiotic; and (ii) they are generally known to possess irritation potential.

Non-limiting examples of classes of non-ionic surfactants that are undesirable according to the present invention include: (i) polyoxyethylene sorbitan esters (polysorbates), such as polysorbate 20, polysorbate 40, polysorbate 60 and polysorbate 80; (ii) sorbitan esters, such as sorbitan monolaurate and sorbitan monooleate; (iii) polyoxyethylene fatty acid esters, such as, PEG-8 stearate, PEG-20 stearate, PEG-40 stearate, PEG-100 stearate, PEG-150 distearate, PEG-8 laurate, PEG-10 laurate, PEG-12 laurate, PEG-20 laurate, PEG-8 oleate, PEG-9 oleate, PEG-10 oleate, PEG-12 oleate, PEG-15 oleate and PEG-20 oleate; (iv) PEG-fatty acid diesters; (v) polyethylene glycol (PEG) ethers of fatty alcohols; (vi) glycerol esters, such as glyceryl monostearate, glyceryl monolaurate, glyceryl monopalmitate and glyceryl monooleate; (vii) PEG-fatty acid mono- and di-ester mixtures; (viii) polyethylene glycol glycerol fatty acid esters; (ix) propylene glycol fatty acid esters; (x) mono- and diglycerides; (xi) sugar esters (mono-, di- and tri-esters of sucrose with fatty acids) and (xii) PEG alkyl phenols.

As mentioned above, in the context of the present invention, while fatty alcohols, fatty acids and certain waxes are somewhat amphiphilic, these substances are not effective as stand-alone surfactants that can stabilize an emulsion, let alone foamable emulsion compositions, because of their very weak emulsifying capacity and further due to their weak foaming capacity on their own.

They are occasionally used in a supporting role as co-emulsifiers, i.e., in combination with a standard surfactant but are commonly used as thickeners and have successfully been used as foam adjuvants to assist customary surfactants to boost foam quality and stability. For the purposes of forming an emulsion they are usually regarded as an oil and thus have a “required” HLB value for the purpose of determining what standard surfactant might be appropriate to use with the oil phase.

Generally, surfactants are known to possess irritation potential. One way to try and reduce or minimize potential irritation and drying of the skin or mucosa due to surfactants and their repeated use, especially when formulations are to be left on the skin or mucosa rather than being washed off, is to use essentially or primarily nonionic surfactants at significant concentrations although preferably below 5%. The current breakthrough of identifying formulations which produce gels and quality breakable foam yet omitting customary surfactants from a composition may contribute to improved tolerability of such a composition and can be an important advantage. This is especially so when a formulation is to be applied to a very sensitive target site, and particularly so on a repeated basis.

In certain embodiments, the composition is free of customary surfactants, or “surfactant-free” and in certain embodiments the foamable composition is substantially free of customary surfactants, or “substantially surfactant-free”.

In certain embodiments, the composition is free or substantially free of an ionic surfactant. In certain embodiments, the composition is free or substantially free of a zwitterionic surfactant. In certain embodiments, the composition is free or substantially free of a non-ionic surfactant.

In one or more embodiments, the composition is substantially alcohol-free, i.e., free of short chain alcohols having up to 5 carbon atoms in their carbon chain skeleton. In other embodiments, the composition comprises less than about 5% by weight final concentration of short chain alcohols, for example, less than 2% by weight, or less than 1% by weight. In certain embodiments, the composition is free or substantially free of ethanol, propanol, butanol and

### Incompatible Excipients and Undesirable Excipients

In certain embodiments, the composition is free of one or more of a petrolatum, surface active agents, protic solvents, certain polar aprotic solvents, isopropyl myristate, polyethylene gelling agents, polyethylene homopolymers, polyethylene copolymers, selenium derivatives and silicone thickening agents; and in certain embodiments, the foamable composition is substantially free of such excipients. In the context herein, the term “substantially-free” relates to a composition that contains a total of less than about 0.4% of a petrolatum, surface active agents, protic solvents, certain polar aprotic solvents isopropyl myristate, polyethylene gelling agents, polyethylene homopolymers, polyethylene copolymers, selenium derivatives and silicone thickening agents cumulatively. Preferably, the composition comprises less than about 0.2% of two or more or all thereof by weight of petrolatum, surface active agents, protic solvents, certain polar aprotic solvents isopropyl myristate, polyethylene gelling agents, polyethylene homopolymers, polyethylene copolymers, selenium derivatives and silicone thickening agents cumulatively or, less than about 0.1% individually, or of two or more, or all thereof cumulatively.

### Tetracycline Antibiotics

The primary active agent in accordance with the present invention is a tetracycline compound (herein “a tetracycline” or “tetracyclines”). The tetracyclines are characterized by a carbon skeleton composed of four linearly fused six-membered carbon rings (octahydrotetracene-2-carboxamide Skeleton). They are defined as “a subclass of polyketides having an octahydrotetracene-2-carboxamide skeleton”. They are collectively known as “derivatives of polycyclic naphthacene carboxamide”.

Non-limiting examples of tetracyclines, include the naturally-occurring Tetracycline, Chlortetracycline, Oxytetracycline and Demeclocycline, the semi-synthetic Doxycycline, Lymecycline, Meclocycline, Methacycline, Minocycline, Rolitetracycline, Chlorotetracycline and Tigecycline.

The tetracyclines can be present in a free base form a hydrate form, a salt form or a complex form. For example, minocycline can be present as the base form, as well as a hydrate or a hydrochloride salt.

Notably, various tetracyclines have different hydrophilic/hydrophobic characters. For example, the Log Kp (log of the of distribution constant at pH 7.0; buffer/CHCl₃) is 1.91, which means that it is highly hydrophilic. The Log Kp of Doxycycline is 0.2; and the Log Kp of Minocycline is −1.6, which stands for hydrophobic character of this compound (see Leive L et al, “Tetracyclines of various hydrophobicities as a probe for permeability of Escherichia coli outer membrane”, Antimicrobial Agents and Chemotherapy 1984:25, 539-544). Whilst any tetracycline compound is suitable as an active agent according to the present invention, there is preference to tetracycline compounds which are more hydrophobic. Thus, in an embodiment of the present invention the active agent is selected as one that has Log Kp equal to, or lower than about 0.2.

In an embodiment, the tetracycline antibiotic is hydrophobic due to the fact that it does not comprise any hydroxy group at Carbons 5, 6, and 7.

In certain embodiments, the tetracycline is selected from the group consisting of doxycycline and minocycline; and in a certain embodiment the tetracycline is minocycline.

According to the present invention, the tetracycline is employed in an amount ranging from about 0.001% to about 10%; or in an amount ranging from about 0.025% to about 6%; or in an amount ranging from about 0.1% to about 3%, by weight of the foamable composition.

In one or more embodiments the concentration of minocycline is in a range between about 0.1% to about 10% by weight (e.g., about 0.1% to about 8% by weight, about 0.1% to about 5% by weight, about 0.1% to about 3% by weight, about 0.1% to about 2% by weight, about 0.1% to about 1% by weight, about 0.1% to about 0.75% by weight, about 0.1% to about 0.5% by weight, about 0.1% to about 0.25% by weight, about 0.25% to about 10% by weight, about 0.5% to about 10% by weight, about 1% to about 10% by weight, about 2% to about 10% by weight, about 4% to about 10% by weight, about 6% to about 10% by weight, about 7% to about 10% by weight, about 8% to about 10% by weight, about 0.5% to about 2.0% by weight, about 0.75% to about 1.5% by weight, about 1% to about 3% by weight, about 1% to about 4% by weight, and about 2% to about 6% by weight). In some embodiments, the concentration of minocycline is at least about 0.05% by weight, is at least about 0.1% by weight, at least about 0.5% by weight, at least about 1% by weight, at least about 2% by weight, at least about 4% by weight, at least about 6% by weight, at least about 8% by weight or at least about 10% by weight.

The tetracycline in accordance to the present invention is insoluble or is partially soluble in the whole composition and all or part thereof is suspended. It is known that every chemical compound has different solubility in different solvents or compositions, and therefore it is not possible to provide a general list compounds that are not soluble or partially soluble or suspended in the composition. However, any tetracycline active agent, as exemplified herein, is suitable as insoluble or partially soluble or suspended, if visual or microscopic observation demonstrates crystals or particles of such active agent in the oleaginous composition.

### Water Activity/Aw

The term “water activity” as used herein, represents the hygroscopic nature of a substance, or the tendency of a substance to absorb water from its surroundings. Microorganisms require water to grow and reproduce, and such water requirements are best defined in terms of water activity of the substrate. The water activity of a solution is expressed as Aw=P/Po, where P is the water vapor pressure of the solution and Po is the vapor pressure of pure water at the same temperature. Every microorganism has a limiting Aw, below which it will not grow; e.g., for Streptococci, Klebsiella spp, Escherichia coli, Clostridium perfringens, and Pseudomonas spp, the Aw value is 0.95. Staphylococcus aureus is most resistant and can proliferate with an Aw as low as 0.86, and fungi can survive at Aw of at least 0.7. In one or more embodiments, the concentration of the hydrophobic solvent, and/or second rheology modulator in the composition is selected to provide an Aw value selected from the ranges between or of (1) about 0.8 and about 0.9; (2) about 0.7 and about 0.8; and (3) less than about 0.7. Delivering the formulation in a pressurized package does not allow for humidity to be absorbed by the preparation, and therefore, the water free character of the composition is not altered.

When the composition of the present invention is water free, it is hygroscopic and its Aw is low.

In an embodiment, hydrophobic carrier has an Aw value of less than 0.9, or less than about 0.8, or less than about 0.7, or less than about 0.6, and preferably less than about 0.5 which is below the level of microbial proliferation.

### Benefits of the Hydrophobic Tetracycline Composition of the Present Invention

The unique hydrophobic composition, as described above, affords surprising clinical outcome in the treatment of wounds, including infected wounds, as demonstrated in Example 1:

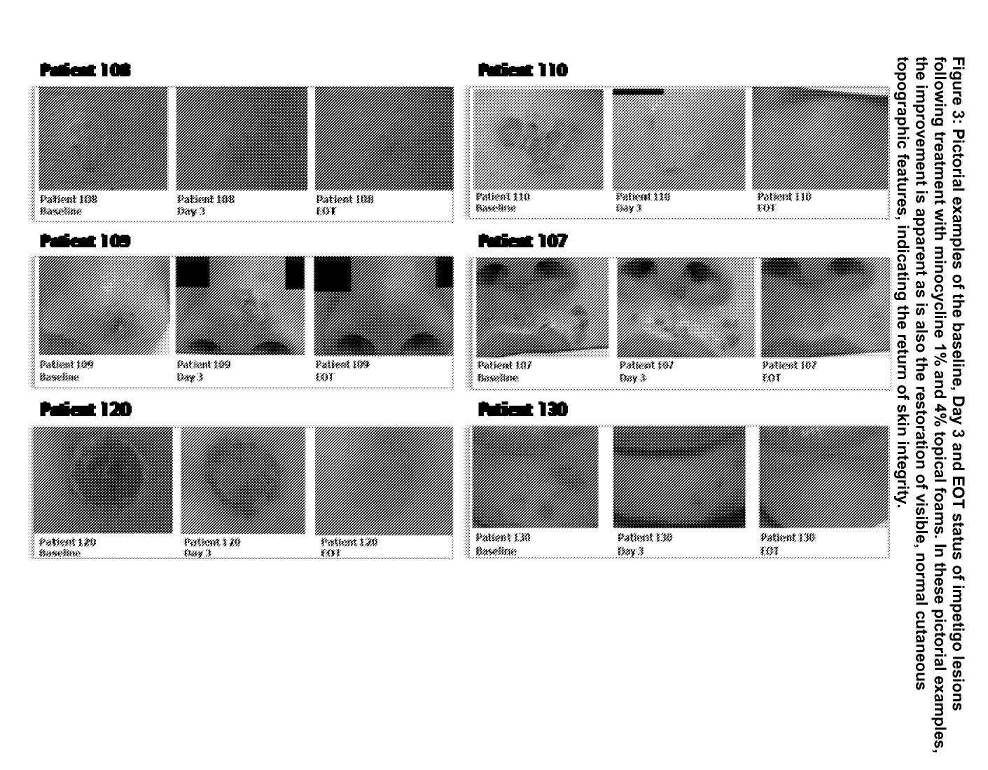

a. Quick Onset of Clinical Effect—Improvement in 80% of the Patients Following Three Days of Treatment.
In a population of patients having a median of 4 infected wounds, in various severity levels, 80% of the patients improved after 3 days of treatment. Improvement was defined as decrease in the number of lesions (meaning that certain lesions seized to exist); or decrease in the size of the lesions; or both. As explained above, the antibiotic effect of the tetracycline antibiotic, within 3 days of treatment, is more rapid than expected. It may be the result of a synergistic combination of the effect of the tetracycline and the vehicle, which is hygroscopic, with low Aw, which contributes to the antibacterial effect of the composition. The healing of the wound, in terms of skin tissue structure, can be initiated once the infection is eradicated. In several cases, after three days of treatment lesions, even deep lesions were cleared and the skin surface was visually healed. Such rapid results are clearly not anticipated by the normal healing timetable, as provided in the above references.
   b. Clinical Success is Achieved in 100% of the Patients Following 7 Days of Treatment.
All 32 patients who participated in the study met the study success criteria following 7 days of treatment. Moreover, about 40% of the lesions were completely cured (100% decrease in lesion size) as exemplified in FIG. 3 (see for example patients No. 109, 110, 120 and 130). After 7 additional days without treatment 69% of the lesions disappeared. Again, such a rate of healing is very rapid and is unexpected in view of the normal healing process, which can take 30 days or longer. While the tetracycline antibiotic is expected to eradicate the bacteria, there was not expectation that it will increase the rate of healing of the skin tissue; and thus these rapid healing results are unexpected and surprising. Without binding to a specific mechanism, it is theorized that the healing effect is obtained by the synergistic combination of the tetracycline antibiotic, which exerts anti-inflammatory and cytokine modulating effects, the hydrophobic oils of the carrier which provide skin conditioning and possibly, the viscosity modulating agents (fatty alcohols and fatty acids), which also can alter inflammation processes.
   c. All Bacteria (Including MRSA) Infections were Cured Following 7 Days of Treatment.
MRSA are stubborn bacteria which are not readily susceptible to antibiotic treatment; however, the treatment using the composition of the present invention eradicated these bacteria in all cases, as detected 7 days after treatment initiation. Again, without binding to a specific mechanism, it is theorized such a strong antibacterial effect is obtained by the synergistic combination of the tetracycline antibiotic, the hydrophobic carrier which is hygroscopic, with low Aw, and the viscosity modulating agents (fatty alcohols and fatty acids), which possess antibacterial properties.
   d. No Scar Formation was Noted, Despite the Accelerated Healing of the Wounds.
Despite the rapid rate of healing process, no scar formation was noted in any of the wounds. This phenomenon is also applicable to burns, acne and rosacea.
Bearing in mind the multiple side effects of oral tetracycline antibiotics, it is further very surprising that such results were not associated with any drug related side effects. Specifically, it is noted that there were no pigmentation, no photo-sensitization, no photo-irritation and no other dermal irritations, which are inherent to tetracyclines appeared. This phenomenon is even more surprising as the concentrations of minocycline found in the skin (in skin penetration studies) is much higher than anticipated for the oral dosage form. Hence, it is theorized that either (i) the known dermal side effects of oral tetracyclines results directly from systemic processes, rather than dermal availability of the drug; or (ii) there is a high manifestation of the anti-inflammatory attributes of the tetracycline, which mitigates the dermal side effects; or (ii) the carrier components, i.e., the hydrophobic oils or the viscosity modifying agents provide a protective anti-inflammatory effect, which mitigates the dermal side effects

### Methods

The compositions provided herein are manufactured according to the methods described in the art and as described Below. Gels are usually packaged in a tube but can also be packaged in any other convenient delivery form including for example, bottles with a pump mechanism or canisters such as bag in can devices where propellant is separate from the gel. Foam formulations are usually packed in a container with an outlet valve. Possible containers and valves are likewise described in the literature as known by those skilled in the art.

### Canisters Filling and Crimping

Each aerosol canister is filled with the pre-foam formulation (“PFF”, i.e., foamable carrier) and crimped with valve using vacuum crimping machine. The process of applying a vacuum will cause most of the oxygen present to be eliminated. Addition of hydrocarbon propellant may, without being bound by any theory, further help to reduce the likelihood of any remaining oxygen reacting with the active ingredient. It may do so, without being bound by any theory, by one or more of dissolving in, to the extent present, the oil or hydrophobic phase of the formulation, by competing with some oxygen from the formulation, by diluting out any oxygen, by a tendency of oxygen to occupy the dead space, and by oxygen occupying part of the space created by the vacuum being the unfilled volume of the canister or that remaining oxygen is rendered substantially ineffective in the formulation.

### Pressurizing & Propellant Filling

Pressurizing is carried out using a hydrocarbon gas or gas mixture. Canisters are filled and then warmed for 30 seconds in a warm bath at 50° C. and well shaken immediately thereafter.

### Tests

By way of non-limiting example the objectives are briefly set out below as would be appreciated by a person of skill in the art.

### Viscosity

Viscosity is measured with Brookfield LVDV-II+PRO with spindle SC4-25 at ambient temperature and 10, 5 and 1 RPM. Viscosity is usually measured at 10 RPM. However, at about the apparent upper limit for the spindle of ˜>50,000 CP, the viscosity at 1 RPM may be measured, although the figures are of a higher magnitude. Unless otherwise stated, viscosity of the pre-foam formulation (PFF) is provided. It is not practical to try and measure the viscosity of the foamable formulation with regular propellants since they have to be stored in sealed pressurized canisters or bottles. In order to simulate the viscosity in the foamable formulations with propellant an equivalent weight of pentane (a low volatile hydrocarbon) is added to and mixed with the pre-foam formulation and left overnight. The viscosity is then measured as above.

### Chemical Stability

The amount of active agent present is analyzed chromatographically in foam released from various pressurized canisters or in the gel or liquid gel. Analysis is carried out at baseline and at appropriate time intervals thereafter. The canisters are typically stored in controlled temperature incubators at one or more of 5° C., 25° C., 40° C. and 50° C. At appropriate time intervals canisters are removed and the amount of active agent in the foam sample is measured.

### Microbiological Tests

Microbial load: Testing was performed according to EP 2.6.12 and 2.6.13 as described in the European Pharmacopea.

Preservative efficacy: Testing was performed according to USP <51> and EP 5.6, 2007 5.1.3. as described in the European and US Pharmacopea.

The test consists of challenging the product with specified microorganisms, storing the inoculated preparations at a prescribed temperature, removing the inoculated samples at specified intervals of time and counting the number of viable organisms in the withdrawn samples using a plate-count procedure. Formulations were challenged by introducing the following microorganisms:
Escherichia coli (ATCC no. 8739)
Staphylococcus aureus (ATCC no. 6538)
Pseudomonas aeruginosa (ATCC no. 9027)
Candida albicans (ATCC no. 10231)
Aspergillus niger (ATCC no. 16404)

The number of colony-forming units (cfu/g) determined at each incubation time point was compared to the number of cfu/g measured in non-inoculated control samples. In order to verify that the samples tested are free of microbial contaminants, the microbial load (base-line) in the samples was determined prior to preservative efficacy testing. Study results are expressed as the number of surviving microorganisms (cfu/g).

Water Activity (Aw): The test for water activity was performed on pre-foam formulation samples introduced into the measuring cell of a PAWKIT water activity meter from DECAGON.

In-vitro effect on microbial growth: The tested microorganism is grown on Tryptic Soy Agar Slants. After incubation, the bacteria is harvested using sterile buffer phosphate pH 7.0, to obtain a microbial count of about 10⁴ cfu/ml. 0.2 ml of the above suspension is spread on Letheen Agar plate and put aside to dry for 20 minutes at room temperature. A sterile disc of 6 mm diameter which has been soaked in 10 μl of the tested antibacterial pre-foam-formulation (PFF) is put on the microbial film, the plate is incubated at 35° C. for 1-2 days. A control experiment is also performed where no antibacterial material is put on the sterile discs. Antimicrobial activity of the tested material inhibits growth of the microorganism around the disc, leaving a transparent zone around it. The diameter of the inhibition zone is measured in mms.

### Compatibility

Active agent is incubated with various excipients individually at one or more temperatures and at different ratios of active agent to a single excipient for a certain fixed period or to the point where degradation was suspected. The period can be for example 3 or 7 or 14 or 21 or 28 days or longer. Visual inspection is a criterion for indication of compatibility. Any change of color indicates oxidation or degradation. For example, the color of an intact MCH suspension is a pale yellow; and a change of color e.g., to dark orange, red, green, brown and black, indicates oxidation or degradation. Tests are also carried out with combinations of excipients.

### Color/Pigmentation

### Part A—Color Change

Samples of formulations are observed and then incubated e.g. during 3 months at 25° C., 30° C. and 40° C. Following this period the foam product is actuated and color is observed, and a change, if any, is noted.

### Part B—Pigmentation

Samples are applied to fair healthy human skin to observe whether any skin pigmentation occurs. The skin is observed prior to and 30 seconds following application.

### General Manufacturing Procedures for a Gel or a Foam

The following procedures are used to produce gel or foam samples, in which only the steps relevant to each formulation are performed depending on the type and nature of ingredients used.

Step 1: Hydrophobic solvents such as mineral oils are mixed at room temperature. Others solvents such as silicones, if present, are added at room temperature under mixing until formulation homogeneity is obtained.

Step 2: The formulation is warmed to 70-80° C. and solid compounds such as fatty alcohols, fatty acids and waxes are added and mixed until complete dissolution.

Step 3: The formulation is cooled down to 30-40° C. and active agents such as tetracyclines are added under mixing until formulation homogeneity is obtained.

Step 4: For gel compositions, the formulation is packaged in suitable containers. For foamable compositions, the formulation is packaged in aerosol canisters which are crimped with a valve, pressurized with propellant and equipped with an actuator suitable for foam dispensing. Optionally, a metered dosage unit can is utilized, to achieved delivery of desirable and/or repeatable measured doses of foam.

Step 5: For foamable compositions, pressurizing is carried out using a hydrocarbon gas or gas mixture. Canisters are filled and then warmed for 30 seconds in a warm bath at 50° C. and well shaken immediately thereafter.

Step 6: The canisters or containers are labeled.

### Example 1

### Randomized, Double-Blind, Multicenter Two Strength Phase 2 Clinical Trial to Assess the Efficacy and Safety of Topical Minocycline Foam in the Treatment of Impetigo in Children

### Materials and Methods

### a) Study Medication

The study medication was supplied as a topical foam comprising minocycline topical at one of two different concentrations (strengths): a lower concentration of 1% by weight and higher concentration of 4% by weight of the formulation. The composition of theses preparations is provided in Table 1. The foam was provided in aluminum aerosol canisters, mounted with a valve and actuator. Each canister contained 25 g of minocycline formulation and 3 g of propellant. Upon actuation of the canister an aliquot of quality foam was released.

TABLE 1
Composition of minocycline hydrochloride (MCH)
Formulations (quantities/100 g preparation)

| Ingredient | MCH 1% | MCH 4% |
|---|---|---|
| Soybean oil | 50.00 | 50.00 |
| Coconut oil | 23.60 | 23.60 |
| Cyclomethicone | 5.00 | 5.00 |
| Light mineral oil | 4.44 | 1.11 |
| Cetostearyl alcohol | 3.50 | 3.50 |
| Stearic acid | 3.00 | 3.00 |
| Myristyl alcohol | 2.50 | 2.50 |
| Hydrogenated castor oil | 2.00 | 2.00 |
| Beeswax | 2.00 | 2.00 |
| Stearyl alcohol | 1.50 | 1.50 |
| Behenyl alcohol | 1.10 | 1.10 |
| Aerosil R 972 (modified silica) | 0.25 | 0.25 |
| Minocycline HCl (micronized) | 1.11 | 4.44 |
| Total | 100.00 | 100.00 |
| Propellant AP-70 (mixture of | 12.00 | 12.00 |
| propane + butane + isobutene) | | |

### b) Clinical Study Design

The protocol and informed consent forms were approved by each clinical site's local Ethics Committee (EC) and the Israel Ministry of Health prior to study initiation. To be eligible for the study, the subject's parent or legal guardian was required to sign a written informed consent document and have been willing and able to comply with the requirements of the protocol. Children aged 2 years and older with at least two impetigo lesions were enrolled and randomized into a parallel group study, testing the two different strengths (1% and 4%) of the study medication.

Treatment was administered topically two times a day (BID) for 7 days to all subjects. Patients were instructed to shake the canister before use, dispense a small amount of foam and apply it as a thin layer on the involved skin. A target total of thirty two subjects were enrolled and randomized with sixteen in each treatment group. The study included four scheduled study visits: Day 1 (Visit 1—Baseline)—screening and treatment initiation; Day 3 (±1)—(Visit 2—Interim visit) with efficacy and safety assessment; Day 7 (±1)—(Visit 3—End of Treatment (EOT)); and, Day 14 (±2) (Visit 4—Follow-up (F/U)). Clinical and bacteriological assessments and efficacy evaluations were done at Baseline, EOT and F/U.

### c) Statistical Methodology

All measured variables and derived parameters were tabulated by descriptive statistics. Descriptive statistics summary tables included sample size, absolute and relative frequency of categorical variables and sample size, arithmetic mean, standard deviation, median, minimum and maximum for means of continuous variables per group.

The Paired T-test was applied for testing differences between baseline assessment and all the post baseline assessments for sum of total area of all lesions within groups, and for efficacy presentation parameters of all lesions within groups.

The Chi-square test was applied for testing the statistical significance of the differences in frequency of categorical variables between the study groups.

95% Confidence Interval (CI) was calculated for the calculated proportions of the main efficacy variables using a binomial proportion for one-way tables.

All tests applied were two-tailed, and p value of 5% or less was considered statistically significant. The data was analyzed using the SAS® version 9.1 for Windows (SAS Institute, Cary N.C.).

### d) Clinical and Bacteriological Response to Treatment

The success criteria (clinical success, clinical failure and bacteriological success) were those specified in the registration trials for the recently approved Altabax as detailed above.¹ Regarding bacteriological response, if after baseline there were no exudates and/or if samples were not taken because the lesion were cleared, such cases were considered a clinical success, pathogen eradication was presumed and the subject was considered a bacteriological success. ¹ Oranje A P, Chosidow O, Sacchidanand S, Todd G, Singh K, Scangarella N, Shawar R, Twynholm M; Topical retapamulin ointment, 1%, versus sodium fusidate ointment, 2%, for impetigo: a randomized, observer-blinded, noninferiority study. Dermatology. 2007; 215(4):331-40.

In addition to clinical response and bacteriological response, the following individual efficacy parameters were also recorded:
Cure, as determined by the Investigators during the study referred to full recovery of the lesions, as observed visually.
Lesion count and area.
Additional signs and symptoms, including erythema, dryness, exudation, peeling, burning, itching and pain (exudation, burning, itching and pain are most relevant to the severity of impetigo). These symptoms were graded from 0 to 3, where 0=none, 1=mild, 2=moderate and 3=severe.

### e) Clinical Microbiology Methods

The microbiology testing of the clinical samples was performed by using culture swabs (Amies) obtained from the target lesion for each study patient, at Days 1, 7 and 14. The patient samples were forwarded to a single microbiology laboratory, at the American Medical Laboratories-AML Israel for processing. All culture swabs were processed the same day that they were collected. Each specimen was aerobically plated into Orientation Agar, Blood Agar (BAP), CDC and thioglycolate. Culture plates were incubated up to 48 hours at 35° C., and then examined for colony morphology consistent with S. aureus and S. pyogenes. Identification of S. aureus and/or S. pyogenes colonies included the following tests: catalase, coagulase (Staphitect, Oxoid), Streptococcal grouping kit (Oxoid). Further identification and sensitivity testing was performed using the MicroScan WalkAway (Siemens) auto analyzer, including oxacillin for S. aureus.

### f) Safety and Tolerability

Safety and tolerability were determined for all randomized patients by the investigator at each visit. All adverse experiences were classified by the investigator as either unrelated; unlikely related; suspected or probably related to the study drug.

### g) Satisfaction

At study visits 3 and 4 (EOT and F/U), the patients' parents filled out a questionnaire regarding treatment satisfaction.

### Results

### a) Study Population

The study was conducted at three centers. A total of thirty-two patients with clinically diagnosed impetigo were randomized to two groups with sixteen patients in each group. One group received the 1% minocycline foam and the other group received the 4% minocycline foam. The study was randomized, and neither the investigators nor the patients and their parents or legal guardian knew which strength of medication was dispensed.

Table 2 summarizes the primary characteristics of the study population and the attendance profile in each study group.

TABLE 2
Patient demographics

| | 1% | 4% | All |
|---|---|---|---|
| Patients randomized | 16 | 16 | 32 |
| Age, years | | | |
| Mean (SD) | 5.9 | 5.6 | 5.8 |
| Range | 2-15 | 3-14 | 2-15 |
| Sex (male/female) | 10/16 | 9/7 | 19/13 |
| Patients who attended Day 3 | 16 | 14 | 30 |
| Patients who attended EOT | 13 | 11 | 24 |
| Patients who attended F/U | 12 | 8 | 20 |
| Patients withdrawn | 4 | 8 | 12 |
| Reasons for withdrawal before F/U | | | |
| Protocol violation | 2 | 3 | 5 |
| Lost to follow-up | 2 | 4 | 6 |
| Withdrew consent | 0 | 1 | 1 |

### b) Efficacy—Baseline Severity

Table 3 provides the baseline severity parameters. The mean number of lesions at Baseline was 4.1 and 3.8 in the 1% and 4% minocycline groups respectively, and the respective median numbers of lesions were 4 and 3.5 in the 1% and 4% minocycline groups respectively.

Notably, the severity of the patients in this study was higher than the severity of patients in the studies conducted with Retapamulin (“the majority of patients in both treatment groups presented with only one impetigo lesion”; median=1). The most common primary lesion site was the face.

Staphylococcus aureus was the most frequently isolated pathogen in the study (56% of isolates in the 1% minocycline group and 75% of isolates from the 4% minocycline group). 34% of the evaluable patients presented isolates of MRSA resistant pathogen.

There was no statistically significant difference between the two groups at baseline with respect to the number and size of lesions, infecting organisms, and the score for exudates, pain, erythema, peeling, dryness and burning. The mean itching score was higher in the 4% minocycline group.

### c) Efficacy—Clinical Response

Clinical response was measured in the course of treatment (Day 3±1), at the end of treatment (EOT) (Day 7±1) and 1 week post EOT (Day 14±2) by assessing the number of lesions, their respective sizes and clinical presentations.

The clinical response rates in the PPC population are summarized in Table 4. Clinical success was demonstrated in both of the two groups among clinical per-protocol (PPC) population at Day 3: being 81% and 79% in the 1% and 4% minocycline groups, respectively. The clinical success at EOT was 92% and 100% in the 1% and 4% minocycline groups; respectively; and, at FU, clinical success was 100% in both groups. As demonstrated in Table 4, the change from baseline was statistically significant in both study groups at Day 3 and the subsequent EOT and F/U visits. No significant differences in overall efficacy were found between the 1% and 4% groups.

TABLE 3
Primary severity parameters at baseline

| | | | | | P value |
|---|---|---|---|---|---|
| | | | | | (1% vs. |
| | 1% | | 4% | | 4%) |
| N | 16 | | 16 | | |
| Mean No. of lesions (SD) | 4.1 | (1.3) | 3.8 | (1.5) | 0.619 |
| Median No. of lesions | 4.0 | | 3.5 | | |
| Total No. of lesions per | 65 | | 61 | | |
| group | | | | | |
| Mean lesions area (SD) | 2.73 | (1.53) | 3.24 | (2.55) | 0.497 |
| Median lesions area | 2.64 | | 2.59 | | |
| No. of patients with micro- | | | | | |
| biologically confirmed infec- | | | | | |
| tion | | | | | |
| Staphylococcus aureus | 9 | | 12 | | 0.264 |
| Streptococcus pyogenes | 6 | | 7 | | 0.719 |
| MRSA | 4 | | 7 | | 0.264 |
| Other | 4 | | 1 | | 0.144 |
| Mean exudation score (SD) | 0.45 | (0.55) | 0.52 | (0.48) | 0.716 |
| Mean itching score (SD) | 0.26 | (0.35) | 0.77 | (0.72) | 0.015 |
| Mean pain score (SD) | 0.71 | (0.76) | 0.58 | (0.68) | 0.613 |
| Mean erythema score (SD) | 0.81 | (0.62) | 0.61 | (0.64) | 0.371 |
| Mean peeling score (SD) | 0.22 | (0.30) | 0.25 | (0.42) | 0.855 |
| Mean dryness score (SD) | 1.68 | (0.72) | 1.77 | (0.77) | 0.745 |
| Mean burning score (SD) | 0.03 | (0.07) | 0.10 | (0.29) | 0.306 |

TABLE 4
Clinical Response by visit

| Clinical | 1% | | 4% | | All | |
|---|---|---|---|---|---|---|
| Response | N | % | N | % | N | % |
| Success | 13 | 81.3 | 11 | 78.6 | 24 | 80.0 |
| Visit 2 | | | | | | |
| (Day 3) | | | | | | |
| P-value | <0.001 | <0.001 | <0.001 | <0.001 | <0.001 | <0.001 |
| (Day 3 vs. | | | | | | |
| baseline) | | | | | | |
| Success | 12 | 92.3 | 11 | 100.0 | 23 | 95.8 |
| Visit 3 | | | | | | |
| (EOT) | | | | | | |
| P-value | <0.001 | <0.001 | <0.001 | <0.001 | <0.001 | <0.001 |
| (EOT vs. | | | | | | |
| baseline) | | | | | | |
| Success | 12 | 100.0 | 8 | 100.0 | 20 | 100.0 |
| Visit 4 | | | | | | |
| (F/U) | | | | | | |
| P-value | <0.001 | <0.001 | <0.001 | <0.001 | <0.001 | <0.001 |
| (EOT vs. | | | | | | |
| baseline) | | | | | | |

### d) Efficacy—Bacteriological Response

Table 5 summarizes the occurrence of bacterial isolates in the study patients at Baseline (B), EOT and F/U.

The majority of the infections in both groups were caused by S. aureus (21/28, 75%) of which approximately 40% were MRSA, as shown in Table 5. The total number of bacterial isolates at baseline in the 1% minocycline group was 20, which decreased to 3 at EOT, representing 85% bacteriological success. The total number of bacterial isolates at baseline in the 4% minocycline group was 27, which decreased to 7 at EOT, representing 74% bacteriological success. The respective bacteriological success rates at F/U were 85% in the 1% minocycline group and 85% in the 4% minocycline group.

Notably, the bacteriological success rate for MRSA infections was 100% and there was no recurrence observed at F/U.

TABLE 5
The occurrence of bacterial isolates in the study PPB patients
at Baseline (B), EOT and F/U - number of patients (%)

| | 1% | | | 4% | | |
|---|---|---|---|---|---|---|
| | B | EOT | F/U | B | EOT | F/U |
| Staphylo- | 9 (75.0) | 1 (11.1) | 0 (0) | 12 (75.0) | 1 (12.5) | 2 (40.0) |
| coccus | | | | | | |
| aureus | | | | | | |
| Strepto- | 6 (50.0) | 1 (11.1) | 1 (14.3) | 7 (43.8) | 5 (62.5) | 1 (20.0) |
| coccus | | | | | | |
| pyogenes | | | | | | |
| MRSA | 4 (33.3) | 0 (0) | 0 (0) | 7 (43.8) | 0 (0) | 0 (0) |
| Other | 1 (8.3) | 1 (11.1) | 2 (28.6) | 1 (6.3) | 1 (12.5) | 1 (20.0) |
| Total | 20 | 3 | 3 | 27 | 7 | 4 |
| number of | | | | | | |
| isolates | | | | | | |

### e) Individual Efficacy Parameters

### Cure

The rate of cure, i.e., total absence of lesions or lesions became dry without crust is displayed in Table 6. Notably, there was 46.2% cure rate in the 1% group at EOT and 58.3% cure at F/U.

TABLE 6
Cure of lesions by visit (PPC population)

| | | |
|---|---|---|
| 1% | 4% | All |

| Cure of lesions | N | % | N | % | N | % |
|---|---|---|---|---|---|---|
| Cure at | 0 | 0 | 1 | 7.1 | 1 | 3.3 |
| Visit 2 (Day 3) | | | | | | |
| Cure at | 6 | 46.2 | 3 | 27.3 | 9 | 37.5 |
| Visit 3 (EOT) | | | | | | |
| Cure at | 7 | 58.3 | 4 | 50.0 | 11 | 55.0 |
| Visit 4 (F/U) | | | | | | |

### Number of Lesions and Lesion Area

Table 7, details the frequency of lesions per patients at baseline, Day 3, EOT and F/U. In the 1% minocycline group at baseline most of the patients (about 93.8%) had 3 or more lesions (37.5% with 3 lesions and 56.3% with 4 or more lesions). At EOT this number was halved (46.2%) and at F/U only 8.3% of the patients had more than 3 lesions.

Table 8 accounts for the total number of lesions in each dosage group (at Baseline, Day 3, EOT and F/U) and provides the number of lesions that disappeared (Size=0). It shows that the total number of lesions decreased dramatically from baseline to EOT and F/U in both minocycline groups. Table 8 further demonstrates that these changes at EOT and F/U were statistically significant in both 1% and 4% minocycline groups.

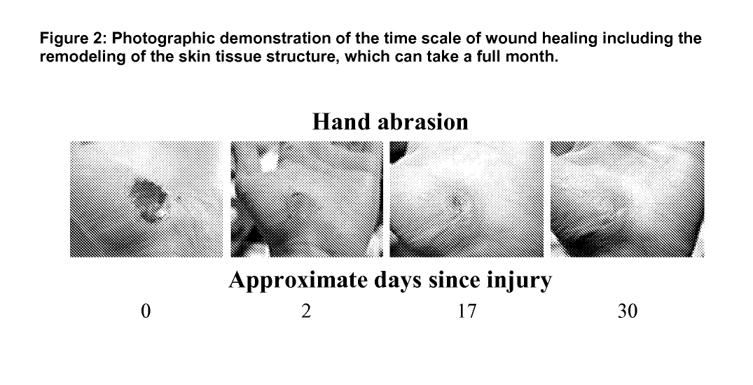

Table 9 provides the mean total area of lesions per patient at baseline and during the subsequent study visits and the mean change of area from baseline in each study visit. As shown in Table 9, at Day 3, the area decreased 26% and 23% in the 1% and 4% minocycline groups, respectively; and, this change from baseline was statistically significant. The subsequent decreases in area were 55% and 47% at EOT in the 1% and 4% minocycline groups, respectively, and 86% and 59% at F/U in the 1% and 4% minocycline groups, respectively. FIG. 2 depicts the mean total area of all lesions (per patient) in PPC population as demonstrated in Table 9.

### Exudate, Itch, Pain and Erythema

Patients were evaluated for seven signs and symptoms: erythema, dryness, exudation, peeling, burning, itching and pain on a scale of 0 to 3: 0=absent, 1=mild, 2=moderate, 3=severe (Tables 10a, 10b and 10c). Exudation (the principal sign of active infection), burning, itching and pain are most relevant to the severity of impetigo.

The decrease in exudation scores from baseline to Day 3 in both the 1% and 4% minocycline groups was clinically and statistically significant. The exudation score further decreased at EOT and F/U.

At EOT and F/U in the 1% minocycline group the decrease in the severity signs and symptoms of erythema, dryness, exudation, itching and pain were statistically significant. In the 4% group dryness, exudation and pain at EOT and F/U were statistically significant. The decrease in erythema score was significant at F/U.

The proportion of subjects who had a score of 0 for blistering at F/U was 100% and 94% for the 1% and 4% groups respectively. The proportion of subjects who had a score of 0 for exudate at F/U was 83.3% and 75% for the 1% and 4% groups respectively.

TABLE 7
Frequency of lesions per patient

| No. of | Baseline | | | | Day 3 | | | | EOT | | | | F/U | | | |
|---|---|---|---|---|---|---|---|---|---|---|---|---|---|---|---|---|
| Lesions | 1% | | 4% | | 1% | | 4% | | 1% | | 4% | | 1% | | 4% | |
| per patient | N | % | N | % | N | % | N | % | N | % | N | % | N | % | N | % |
| 2 | 1 | 6.3 | 4 | 25.0 | 2 | 12.5 | 4 | 28.6 | 2 | 15.4 | 2 | 18.2 | 3 | 25.0 | 1 | 12.5 |
| 3 | 6 | 37.5 | 4 | 25.0 | 5 | 31.3 | 3 | 21.4 | 2 | 15.4 | 2 | 18.2 | 0 | 0 | 1 | 12.5 |
| ≧4 | 9 | 56.3 | 8 | 50.0 | 9 | 56.3 | 6 | 42.9 | 4 | 30.8 | 3 | 27.3 | 1 | 8.3 | 2 | 25.0 |

N = No. of patients with the specified lesions number

TABLE 8
Total number of lesions at Day 3, EOT
and F/U that disappeared (Size = 0)

| | 1% | | 4% | | All | |
|---|---|---|---|---|---|---|
| | N | % | N | % | N | % |
| Baseline | 0 | 0 | 0 | 0 | 0 | 0 |
| Visit 2 - Day 3 | 2 | 3.1 | 6 | 11.3 | 8 | 6.7 |
| P-value (Day 3 | 0.333 | | 0.082 | | 0.050 | |
| vs. baseline) | | | | | | |
| Visit 3 - EOT | 20 | 39.2 | 17 | 38.6 | 37 | 38.9 |
| P-value (EOT | 0.003 | | 0.018 | | <0.0001 | |
| vs. baseline) | | | | | | |
| Visit 4 - F/U | 42 | 79.2 | 22 | 61.1 | 64 | 71.9 |

| | | | |
|---|---|---|---|
| P-value (F/U | <0.0001 | 0.015 | <0.0001 |
| vs. baseline) | | | |

N = No. of lesions that disappeared

TABLE 9
Decrease of total area of all lesions (per patient) (PPC population)

| 1% | | | 4% | | | All | | |
|---|---|---|---|---|---|---|---|---|
| N | Mean | P* | N | Mean | P* | N | Mean | P* |

Mean area (cm²)

| | | | | | | |
|---|---|---|---|---|---|---|
| Baseline | 16 | 2.73 cm² | 16 | 3.24 cm² | 32 | 2.98 cm² |
| Visit 2 | 16 | 2.03 cm² | 14 | 2.30 cm² | 30 | 2.15 cm² |
| Visit 3 | 13 | 1.37 cm² | 11 | 1.65 cm² | 24 | 1.50 cm² |
| Visit 4 | 12 | 0.48 cm² | 8 | 0.67 cm² | 20 | 0.56 cm² |

Decrease of lesion area per patient (cm²)

| | | | | | | | | | |
|---|---|---|---|---|---|---|---|---|---|
| Visit 2 | 16 | −0.70 cm² | 0.012 | 14 | −0.73 cm² | 0.002 | 30 | −0.71 cm² | <.001 |
| Visit 3 | 13 | −1.51 cm² | <.001 | 11 | −1.51 cm² | <.001 | 24 | −1.51 cm² | <.001 |
| Visit 4 | 12 | −2.34 cm² | <.001 | 8 | −1.92 cm² | 0.002 | 20 | −2.17 cm² | <.001 |

Decrease of lesion area per patient (%)

| | | | | | |
|---|---|---|---|---|---|
| Visit 2 | 16 | −26% | 14 | −23% | 30 |
| Visit 3 | 13 | −55% | 11 | −47% | 24 |
| Visit 4 | 12 | −86% | 8 | −59% | 20 |

*P-value for changes vs. Baseline (paired t-test)

TABLE 10a
Change of severity signs and symptoms
from Day 3 to Baseline (PPC population)

| | 1% | | | 4% | | | All | | |
|---|---|---|---|---|---|---|---|---|---|
| | N | Mean | P | N | Mean | P | N | Mean | P |
| Erythema | 16 | −0.20 | 0.227 | 14 | −0.28 | 0.183 | 30 | −0.24 | 0.065 |
| Dryness | 16 | 0.02 | 0.936 | 14 | −0.06 | 0.831 | 30 | −0.02 | 0.914 |
| Exudation | 16 | −0.16 | 0.030 | 14 | −0.41 | 0.011 | 30 | −0.27 | 0.001 |
| Peeling | 16 | 0.14 | 0.220 | 14 | 0.30 | 0.871 | 30 | 0.21 | 0.012 |
| Burning | 16 | 0.05 | 0.479 | 14 | −0.05 | 0.336 | 30 | 0.00 | 0.928 |
| Itching | 16 | −0.20 | 0.129 | 14 | −0.14 | 0.444 | 30 | −0.17 | 0.111 |
| Pain | 16 | −0.14 | 0.321 | 14 | −0.69 | 0.002 | 30 | −0.39 | 0.003 |

TABLE 10b
Change of Efficacy Presentation from
Visit 3 to Baseline (PPC population)

| | 1% | | | 4% | | | All | | |
|---|---|---|---|---|---|---|---|---|---|
| | N | Mean | P | N | Mean | P | N | Mean | P |
| Erythema | 13 | −0.46 | 0.012 | 11 | −0.23 | 0.442 | 24 | −0.36 | 0.032 |
| Dryness | 13 | −0.96 | 0.020 | 11 | −1.05 | 0.011 | 24 | −0.99 | <.001 |
| Exudation | 13 | −0.24 | <.001 | 11 | −0.42 | 0.021 | 24 | −0.32 | <.001 |
| Peeling | 13 | −0.12 | 0.093 | 11 | 0.17 | 0.179 | 24 | 0.01 | 0.898 |
| Burning | 13 | −0.03 | 0.175 | 11 | 0.00 | — | 24 | −0.02 | 0.170 |
| Itching | 13 | −0.60 | 0.005 | 11 | −0.35 | 0.256 | 24 | −0.48 | 0.006 |
| Pain | 13 | −0.23 | 0.013 | 11 | −0.74 | 0.013 | 24 | −0.46 | 0.002 |

TABLE 10c
Change of Efficacy Presentation from
Visit 4 to Baseline (PPC population)

| | 1% | | | 4% | | | All | | |
|---|---|---|---|---|---|---|---|---|---|
| | N | Mean | P | N | Mean | P | N | Mean | P |
| Erythema | 12 | −0.59 | 0.004 | 8 | −0.31 | 0.016 | 20 | −0.48 | <.001 |
| Dryness | 12 | −1.27 | 0.001 | 8 | −1.17 | 0.021 | 20 | −1.23 | <.001 |
| Exudation | 12 | −0.23 | 0.011 | 8 | −0.48 | 0.004 | 20 | −0.33 | <.001 |
| Peeling | 12 | −0.12 | 0.160 | 8 | 0.11 | 0.290 | 20 | −0.03 | 0.636 |
| Burning | 12 | 0.00 | 0.983 | 8 | 0.00 | — | 20 | 0.08 | 0.983 |
| Itching | 12 | −0.74 | 0.003 | 8 | −0.14 | 0.587 | 20 | −0.50 | 0.007 |
| Pain | 12 | −0.26 | 0.015 | 8 | −0.83 | 0.017 | 20 | −0.49 | 0.002 |

### f) Photographic Examples of Successful Treatment of Impetigo Lesions

FIG. 3 provides pictorial examples of the baseline, Day 3 and EOT status of impetigo lesions following treatment with Minocycline 1% and 4% topical foams. In these pictorial examples, the improvement is apparent as is also the restoration of visible, normal cutaneous topographic features, indicating the return of skin integrity.

### g) Safety and Tolerability

Safety was determined for all randomized patients by interview at each visit. All adverse experiences were judged by the investigator to be not related; possibly related; or related to the study drug.

There were no clinical recurrences and no adverse events in any of the groups. Minocycline topical foam administered twice daily was well-tolerated, with high rates of clinical and microbiological responses for treating impetigo.

### h) Satisfaction Questionnaires

As with other therapeutic regimens, patient compliance is essential in the effectiveness of prescribed antibiotics. With poor compliance, therapeutic goals are less likely to be achieved, resulting in poorer patient outcomes. Poor compliance is associated with deteriorating health, the need for additional consultations, the emergence of bacterial resistance, extra drugs, additional hospital admissions, and increases in direct and indirect costs of healthcare management.

In general, patients are more compliant with simple and less-frequent dosing regimens. Both the dosage schedule and the patient's daily routine should be considered when prescribing antibiotics.² Topical agents may also be more attractive than oral therapy because they reduce the potential for systemic side effects, typically nausea and diarrhea, which are commonly associated with many systemic antibiotics. ² Cockburn J, Gibberd R W, Reid A L, Sanson-Fisher R W. Determinants of non-compliance with short term antibiotic regimens. Br Med J. 1987; 295:814-818.

Satisfaction questionnaires, answered by the patient's parents at EOT, revealed high satisfaction with treatment, as exemplified in Table 11. In the General Satisfaction category a majority of caregivers (more than 55%) in both groups rated the product as “very satisfactory” or “excellent” and a further 33% and 44% in the 1% and 4% minocycline groups respectively rated it as “moderately satisfactory” raising the general level of satisfaction to over 90%. Likewise, in the Usability category 71% of the caregivers in all groups rated the product as “very satisfactory” or “excellent” and a further 24% in all groups rated it as “moderately satisfactory” raising the general level of usability to over 90%. None of the caregivers rated the product as “unsatisfactory”.

TABLE 11
General satisfaction and usability rating, as opined by patients' caregivers at EOT.

| | General satisfaction | | | | | | Usability | | | | | |
|---|---|---|---|---|---|---|---|---|---|---|---|---|
| | 1% | | 4% | | All | | 1% | | 4% | | All | |
| | N | % | N | % | N | % | N | % | N | % | N | % |
| 5 (Excellent) | 4 | 33.3% | 2 | 22.2% | 6 | 28.6% | 1 | 8.3% | 2 | 22.2% | 3 | 14.3% |
| 4 (Very satisfactory) | 3 | 25% | 3 | 33.3% | 6 | 28.6% | 9 | 75% | 3 | 33.3% | 12 | 57.1% |
| 3 (Moderate) | 4 | 33.3% | 4 | 44.4% | 8 | 38.1% | 2 | 16.7% | 3 | 33.3% | 5 | 23.8% |
| 2 (Slight) | 1 | 8.3% | 0 | 0% | 1 | 4.7% | 0 | 0% | 1 | 11.1% | 1 | 4.8% |
| 1 (Unsatisfactory) | 0 | 0% | 0 | 0% | 0 | 0% | 0 | 0% | 0 | 0% | 0 | 0% |

1. Discussion

This is the first clinical study to evaluate the safety and efficacy of topical minocycline in treating impetigo. It shows that topical minocycline foam is a highly effective and convenient new treatment option for impetigo, with early clinical response evident on the first visit after 3 days of treatment:
About 80% clinical success was observed after 3 days of treatment in both groups.
100% clinical success was observed on Day 7 in 4% minocycline group and on Day 14 in the 1% minocycline group.
All MRSA infections were eradicated at EOT (Day 7).
No drug-related side effects were recorded.

These impressive effects were achieved following twice daily topical application of minocyline foam for seven days. In contrast, the currently available topical antibiotics Fucidin (fucidic acid, LEO Pharma) and Bactroban (mupirocin, GSK) require three daily treatments. Comparison of the current study protocol with the studies carried out of Altabax (retapamulin, GSK) shows that the severity of impetigo in the current minocycline foam study was far higher than the respective severity in the Altabax studies—with the median numbers of lesions at Baseline (3.5-4 per patient) being significantly higher than the reported baseline numbers in the retapamulin studies (“the majority of patients in both treatment groups in the retapamulin studies presented with only one impetigo lesion”; median=1).

The effective eradication of MRSA is encouraging and gives rise to curing the patients, as well as protecting the surrounding infants and children from contracting resistant bacterial infections.

The study population comprised pediatric patients, aged 2-15 years old; and, yet, the drug was well tolerated and positively rated for its effect and usability by the patients and their caregivers. Not only was it well-tolerated (meaning it did not cause local AEs), it lead to the rapid reduction of signs and symptoms. Pain reduction and itching reduction make patients more comfortable; and the itch reduction and exudate reduction can minimize the risk for infecting playmates and siblings.

Thus, topical minocycline foam offers a safe and effective alternative to fusidic acid, mupirocin and retapamulin for the topical treatment of impetigo in children. The ease of use, with twice daily dosing, as well as its broad spectrum of activity, the lack of adverse effects and the rapid reduction of signs and symptoms make it an attractive choice. These results warrant additional clinical studies in order to establish the role of topical minocycline foam as a potentially valuable medication for the treatment of acute bacterial skin infections.

### Example 2

### Efficacy and Safety of Topical Minocycline Foam for Treatment of Acne

### Materials and Methods

The forehead of a fourteen year old male subject having acne vulgaris was cleansed, than treated topically with a hydrophobic composition comprising 1% minocycline (the composition is provided in Table 1 of Example 1), at bedtime once daily for 6 weeks. Subject's forehead was photographed at baseline and after 6 weeks of treatment.

### Results

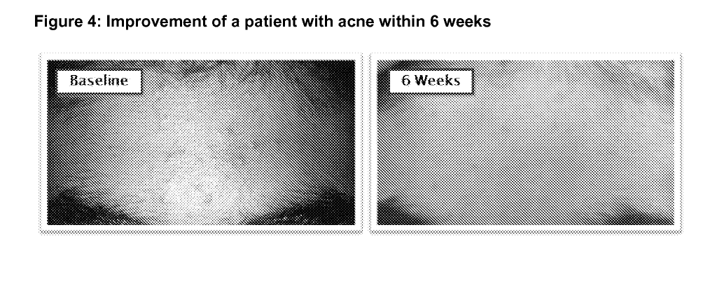

Following six weeks of treatment, in which the hydrophobic composition containing 1% minocycline was applied once daily on the forehead of a fourteen year old male afflicted with acne vulgaris, an unexpected decrease in the number of both inflammatory and non-inflammatory lesions and a significant improvement in the skin condition was observed (See FIG. 4). As shown in FIG. 4, the improvement is apparent as is also the restoration of visible, normal cutaneous topographic features, indicating the return of skin integrity. No systemic adverse events and no dermal adverse events, e.g., skin irritation, photosensitization, pigmentation, erythema, dryness, itching or peeling normally associated with oral antibiotics or other available topical formulation were observed.

The lack of pigmentation and photosensitization is surprising as hyper-pigmentation and photosensitization of the skin are known dermal side effects of oral minocycline. It could be therefore expected that more severe pigmentation and photosensitization would be observed if minocycline is applied directly onto the skin; but this was not the case. FIG. 4 demonstrates full clearance of the lesions in the treated area, while the known effect of oral minocycline is about 50% on inflammatory lesions and no effect on non-inflammatory lesions. These preliminary results may imply that the composition of the present invention is as effective as, or more effective than, the alternatives without untoward reactions.

As acne and acne-rosacea share common etiological features, these findings imply that treatment of rosacea with the hydrophobic tetracycline composition of the present invention should also result in therapeutic benefits.

### Example 3

### Stability of Tetracycline Compositions

The following examples illustrate the chemical stability of minocycline HCl (“MCH”) in hydrophobic formulations, In an accelerated stability study, samples were stored at 40° C., and the concentrations of minocycline HCl were determined by UPLC. The stability test results following 2 months, 3 months, 6 months, 9 months, 12 months, 18 months of storage are shown herein below.

Samples of MCH 1% and 4% were stored at 25° C. and 40° C. in order to test physical and chemical stability.

The use of pressurized glass bottles enables the inspection of formulations for homogeneity in the presence of propellant. Following 18 months of storage at 25° C. the formulation was found to be re-dispersible, i.e., homogeneous following slight shaking.

Storage at 25° C. and 40° C. for 18 months revealed almost no change in the Minocycline concentration. There was practically no degradation of 1% and 4% minocycline following 18 months at 25° C. and also following 9 months at 40° C. These stability results indicate shelf life of more than two years at ambient temperature. These stability results likewise indicate a long shelf life of more than two years at ambient temperature. In one or more embodiments the tetracycline composition has a shelf life of at least 6 months, or at least 9 months, or at least 12 months or at least 15 months, or at least 18 months or at least 21 months or at least 24 months at ambient temperature. In one or more embodiments the tetracycline composition has a shelf life of at least 6 months, or at least 9 months, or at least 12 months or at least 15 months, or at least 18 months or at least 21 months or at least 24 months at 25° C. In one or more embodiments the tetracycline composition has a shelf life of at least 1 month, or at least 3 months, or at least 3 months or at least 6 months, or at least 9 months or at least 12 months 40° C.

TABLE 12
Minocycline content in MCH 1% following storage for
18 months at 25° C. and 40° C.

Minocycline content (% w/w)

| Temp | T = 0 | 3 M | 6 M | 9 M | 12 M | 18 M |
|---|---|---|---|---|---|---|
| 25° C. | 1.001 | NM | 0.986 | 1.007 | 0.972 | 0.959 |
| 40° C. | 1.001 | 1.002 | 0.983 | 0.965 | NM | NM |

TABLE 13
Minocycline content in MCH 4% following storage for
18 months at 25° C. and 40° C.

Minocycline content (% w/w)

| Temp | T = 0 | 3 M | 6 M | 9 M | 12 M | 18 M |
|---|---|---|---|---|---|---|
| 25° C. | 1.012 | NM | 0.998 | 0.998 | 0.972 | 0.925 |
| 40° C. | 1.012 | 0.963 | 1.009 | 0.978 | NM | NM |

1. A method for restoring skin integrity or accelerating the restoration of the integrity of an area of a skin or mucosal lesion comprising broken skin or a damaged mucosa, the method comprising topical application of a hydrophobic gel or foam composition to said skin or mucosal lesion,
wherein the gel or foam composition comprises a tetracycline antibiotic and a carrier comprising about 60% to about 99% by weight of at least one hydrophobic oil.

2. The method of claim 1, wherein the carrier further comprises at least one viscosity-modifying agent, selected from the group consisting of a fatty alcohol, a fatty acid and a wax.

3. The method of claim 1, wherein the tetracycline antibiotic is:
a. a derivative of polycyclic naphthacene carboxamide; or
b. selected from tetracycline, chlortetracycline, oxytetracycline, demeclocycline, doxycycline, lymecycline, meclocycline, methacycline, minocycline, rolitetracycline, chlorotetracycline and tigecycline; or
c. a tetracycline antibiotic having Log Kp equal to, or lower than about 0.2;
d. a tetracycline antibiotic that does not comprise any hydroxy group at Carbons 5, 6, and 7
wherein the tetracycline antibiotic is a free base, or hydrate form, or a salt form or a complex form, or a derivative of said tetracycline antibiotic.

4. The method of claim 3, wherein the tetracycline antibiotic is a doxycycline or a minocycline.

5. The method of claim 2, wherein the tetracycline antibiotic is present in the composition in an amount ranging from about 0.1% to about 10%.

6. The method of claim 2, wherein the application is at least once daily for at least three days.

7. The method of claim 2, wherein the gel is contained in a canister to which is added a propellant and the foam is formed when the composition is released from the canister.

8. The method of claim 1, wherein the broken skin is due to a disorder selected from the group consisting of a wound, a chronic wound, a burn, impetigo, acne, rosacea an inflammation, an ulcer, and a skin disease caused by a bacteria.

9. The method of claim 1, wherein the tetracycline antibiotic is minocycline hydrochloride and wherein the minocycline hydrochloride is present in the composition at a concentration of about 1% by weight, about 4% by weight or in a concentration ranging from about 1% and about 4%.

10. The method of claim 1, wherein the hydrophobic gel or foam composition is applied at a frequency selected from the group consisting of three times daily, twice daily, and once daily; and is administered for a period selected from the group consisting of at least three days, four days, five days, six days, seven days, eight days, nine days, ten days, eleven days, twelve days, thirteen days, and two weeks.

11. The method of claim 1, wherein the hydrophobic foam or gel composition is effective against methicillin-resistant S. aureus bacteria associated disorders.

12. The method of claim 1, wherein the restoration of skin integrity is achieved within seven days.

13. The method of claim 12, wherein the loss of skin integrity was due to impetigo.

14. The method of claim 1, wherein the onset of healing is achieved within three days.

15. The method of claim 2, wherein the hydrophobic gel or foam composition comprises:
a) about 48% to about 51% by weight of soybean oil;
b) about 23% to about 25% by weight of coconut oil;
c) about 4% to about 6% by weight of cyclomethicone;
d) about 0.5% to about 5% by weight of light mineral oil;
e) about 3% to about 4% by weight of cetostearyl alcohol;
f) about 2% to about 4% by weight of stearic acid;
g) about 2% to about 3% by weight of myristyl alcohol;
h) about 1% to about 3% by weight of hydrogenated castor oil;
i) about 1% to about 3% by weight of beeswax;
j) about 1% to about 2% by weight of stearyl alcohol;
k) about 0.5% to about 1.5% by weight of behenyl alcohol;
l) about 0.2% to about 0.5% by weight of modified (fumed) silica; and
m) about 1% or about 4% by weight of minocycline hydrochloride or doxycycline hyclate.
and wherein the tetracycline antibiotic is suspended in the carrier.
